(19) United States
(12) Patent Application Publication
(10) Pub. No.: US 2025/0054583 A1
(43) Pub. Date: Feb. 13, 2025

(54) LEARNING DEVICE, LEARNING METHOD, SCREENING DEVICE, AND SCREENING METHOD

(71) Applicant: ENEOS Corporation, Tokyo (JP)

(72) Inventors: Tasuku ONODERA, Tokyo (JP); Takashi SANO, Tokyo (JP)

(21) Appl. No.: 18/795,399

(22) Filed: Aug. 6, 2024

(30) Foreign Application Priority Data
Aug. 10, 2023 (JP) 2023-131534

(51) Int. Cl. G16C 20/30; G16C 20/70

(52) U.S. Cl. CPC G16C 20/30; G16C 20/70

(57) ABSTRACT

A learning device includes a processor; and a memory storing program instructions that cause the processor to generate a learned model by performing machine learning using a training dataset in which a descriptor of a molecular structure of an adsorbate to be adsorbed to an adsorbent is associated with an interaction index of one or more intermolecular bonds of interest between the adsorbate and the adsorbent.

### CROSS-REFERENCE TO RELATED APPLICATIONS

This patent application is based on and claims priority to Japanese Patent Application No. 2023-131534 filed on Aug. 10, 2023, the entire contents of which are incorporated herein by reference.

### TECHNICAL FIELD

The present disclosure relates to a learning device, a learning method, a screening device, and a screening method.

### BACKGROUND

Attempts have been made to predict predetermined performance such as an adsorptive property of an adsorbate (adsorption substance), such as a molecule or a molecular crystal, to an adsorbent formed of a solid, such as a molecular crystal, a metal, or a ceramic, by utilizing computational chemistry, such as quantum chemical calculation. By using such a method of predicting the performance of the adsorbate, the predetermined performance of the adsorbate can be evaluated without actually producing the adsorbate and performing a test, and therefore, the cost and time required for the discovery, design, production, and the like of the adsorbate that satisfies the predetermined performance can be reduced.

As a method of predicting the performance of the adsorbate by utilizing computational chemistry, for example, a computer device has been proposed that models an interaction between an adsorbate and a metal surface, combines the model with quantum chemical calculation for the adsorbate, and obtains physical properties, such as the energy of the adsorbate itself.

### SUMMARY

According to one aspect of the present disclosure, a learning device includes a processor; and a memory storing program instructions that cause the processor to generate a learned model by performing machine learning using a training dataset in which a descriptor of a molecular structure of an adsorbate to be adsorbed to an adsorbent is associated with an interaction index of one or more intermolecular bonds of interest between the adsorbate and the adsorbent.

According to another aspect of the present disclosure, a learning method includes generating a learning model by using a training dataset in which a descriptor of a molecular structure of an adsorbate to be adsorbed to an adsorbent is associated with an interaction index of one or more intermolecular bonds of interest between the adsorbate and the adsorbent.

According to another aspect of the present disclosure, a screening device includes a processor; and a memory storing program instructions that cause the processor to acquire a prediction target adsorbent and a descriptor of a molecular structure of a prediction target adsorbate to be adsorbed to the prediction target adsorbent; and predict an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.

According to another aspect of the present disclosure, a screening method includes acquiring a prediction target adsorbent and a descriptor of a molecular structure of a prediction target adsorbate to be adsorbed to the prediction target adsorbent; and predicting an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.

### BRIEF DESCRIPTION OF THE DRAWINGS

FIG. 1A and FIG. 1B are explanatory diagrams each illustrating a state in which an adsorbate is adsorbed to an adsorbent;

FIG. 2A and FIG. 2B are explanatory diagrams each illustrating a bonding distance between a molecular crystal and a molecule;

FIG. 3 is a block diagram illustrating a schematic configuration of a learning device according to an embodiment of the present disclosure;

FIG. 4 is a graph indicating an example of a correlation between the interaction index lint and the absolute value of the adsorption energy E_{ads};

FIG. 5 is a diagram illustrating a configuration of a screening system according to the embodiment of the present disclosure;

FIG. 6 is a block diagram illustrating a schematic configuration of a screening device according to a first embodiment;

FIG. 7 is a block diagram illustrating a schematic configuration of a first energy calculation unit;

FIG. 8 is a block diagram illustrating a schematic configuration of a second energy calculation unit;

FIG. 9 is a graph indicating an example of clustering;

FIG. 10 is a block diagram illustrating a schematic configuration of a screening device according to a second embodiment;

FIG. 11 is a block diagram illustrating a hardware configuration of the learning device and the screening device;

FIG. 12 is a flowchart for explaining a learning method according to the embodiment of the present invention;

FIG. 13 is a flowchart illustrating an example of a screening method according to the first embodiment;

FIG. 14 is a flowchart illustrating an example of a first energy calculation step;

FIG. 15 is a flowchart illustrating an example of a second energy calculation step;

FIG. 16 is a flowchart illustrating an example of a screening method according to the second embodiment;

FIG. 17 is a correlation graph between the interaction index Iᵢₙₜ and the absolute value of the adsorption energy E_{ads} in Example 1;

FIG. 18 is a graph plotting the average value of the interaction indexes Iᵢₙₜ and the absolute value of the adsorption energy E_{ads} for each cluster in Example 1;

FIG. 19 is a diagram illustrating a group of molecules of one cluster;

FIG. 20 is a graph indicating a relationship between a calculated value and a predicted value of the interaction index Iᵢₙₜ in Example 2; and

FIG. 21 is a graph indicating a relationship between a molecule and a predicted value of the interaction index Iᵢₙₜ in Example 2.

### DETAILED DESCRIPTION

When an adsorbate having a predetermined adsorptive property is selected by exhaustively screening adsorbates adsorbed to a surface of a specific adsorbent, from a huge number of substances, by using the adsorption energy as an index, a calculation load is large even when only the adsorption energy is calculated. When the quantum chemical calculation, such as the first-principles calculation, is used, it takes a very long time to perform processes such as setting calculation conditions, examining the direction of the adsorbate with respect to the adsorbent, calculating adsorption energy, and calculating a value serving as an index of the strength of adsorption. Therefore, there is a problem that it is not realistic to perform calculation for all adsorbates to be adsorbed to the adsorbent.

According to the present disclosure, the adsorptive property of the adsorbate to the adsorbent can be predicted without directly obtaining the adsorption energy of the adsorbate adsorbed to the surface of the adsorbent.

In the following, embodiments of the present disclosure will be described in detail. Here, in order to facilitate understanding of the description, the same components are denoted by the same reference symbols in the drawings, and the duplicated description thereof will be omitted. Additionally, in the present specification, “to” used to indicate a numerical range indicates that numerical values described before and after “to” are included as a lower limit value and an upper limit value, unless otherwise specified.

### <Learning Device>

A learning device according to the present embodiment will be described. The learning device according to the present embodiment performs learning by using a descriptor of a molecular structure of an adsorbate to be adsorbed on an adsorbent as an explanatory variable, and using a molecular interaction index Iᵢₙₜ of an intermolecular bond of interest between the adsorbate and the adsorbent (hereinafter, simply referred to as the “interaction index Iᵢₙₜ”) as an objective variable, to generate a learned model that predicts an interaction index Iᵢₙₜ from a descriptor of a molecular structure of an adsorbate.

Here, in the present embodiment, the interaction index Iᵢₙₜ may be an index representing the intermolecular bond of interest between the adsorbate and the adsorbent, and can be obtained by, for example, the following equation (1).

$$\begin{matrix}[ Eq . 1 ] & \\ {I}_{int} = \frac{A}{N} {\sum }_{i}^{N} exp ( - \frac{{R}_{i} - {R}_{0}}{{R}_{0}} ) & ( 1 )\end{matrix}$$

(where Iᵢₙₜ is the interaction index, A and R₀ are constants, N is the number of intermolecular bonds of interest at the interface with the adsorbent to which the adsorbate is surface-adsorbed, and Rᵢ is the distance of the intermolecular bonds of the interest at the interface with the adsorbent to which the adsorbate is surface-adsorbed.)

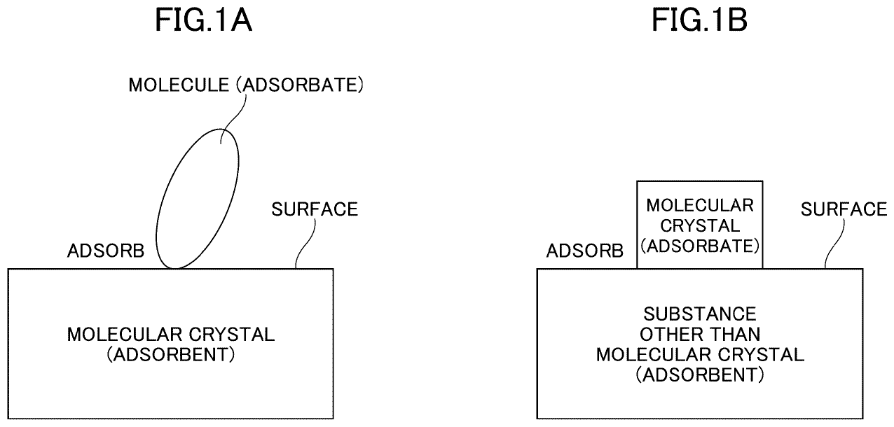

In the present embodiment, the adsorbent is a substance to which the adsorbate is adsorbed, and examples of the adsorbent include a solid substance such as a molecular crystal, a metal, and a ceramic, as illustrated in FIG. 1. The adsorbate is a substance (adsorbed substance) to be adsorbed to the adsorbent, and examples of the adsorbate include a molecule and a molecular crystal, as illustrated in FIG. 1. Either the adsorbent or the adsorbate may be a molecular crystal. The molecule adsorbed to the adsorbent (adsorbed molecule) may be a single molecule, a polymer containing a molecule, or a compound including a polymer. In the present embodiment, the adsorbent is a molecular crystal, and the adsorbate is a molecule adsorbed to the molecular crystal (hereinafter, simply referred to as the molecule).

The molecular crystal generally refers to a substance in which molecules are aggregated and solidified to form a periodic crystal structure. The molecular crystal differs from a general crystal in the type of interaction. The general crystal is mainly formed by an interaction such as a covalent bond, an ionic bond, or a metal bond between atoms. Examples of the general crystals include diamond, sodium chloride, iron, and the like. The molecular crystal is substantially the same as the general crystal in that a covalent bond or the like is a main interaction inside each molecule constituting the molecular crystal, but is different in that the molecular crystal is formed by a relatively weak interaction such as a van der Waals interaction between molecules. Additionally, in addition to the case where the molecular crystal is formed by relatively weak interaction, a case where the molecular crystal is formed by hydrogen bonding, which is non-covalent attractive interaction formed between a hydrogen atom covalently bonded to an atom having a high electronegativity and a lone pair of electrons of nitrogen, oxygen, sulfur, fluorine, or the like disposed in the vicinity thereof, or interaction between a π electron of an aromatic ring or the like and another functional group or the like. Examples of the molecular crystal include carbon dioxide (CO₂) forming dry-ice and molecular iodine (I₂), a crystal of proteins, and thickeners forming a grease used for lubrication of machines.

There are many phenomena in which a molecule constituting another substance is adsorbed to the surface of a molecular crystal as illustrated in FIG. 1A or in which a molecular crystal itself adsorbs to the surface of another substance as illustrated in FIG. 1B. Adsorption phenomena involving substances, such as molecules or molecular crystals, are important phenomena in the medical field, the industrial field, and the like. In the medical field, for example, understanding of a phenomenon in which water contained in a living body, another biomolecule, or a molecule of a drug for a pharmaceutical product is adsorbed to a surface of a protein constituting the living body is an important factor in biology, pharmacy, and the like. The phenomenon that a protein itself is adsorbed to a surface of a material, such as a ceramic or a polymer, is important in designing a material itself of a biomaterial constituting an artificial joint or the like, or the lubricating performance of the material. In the industrial field, a thickener forming a grease used for lubrication of machines may have a crystal structure, and the lubricating state is influenced by the adsorption phenomenon. Therefore, the adsorption phenomenon is important in designing the lubricating performance of a grease and in understanding the lubricating state of the machine.

The surface of such a molecular crystal does not have a large surface energy, unlike, for example, a metal surface, and, microscopically, an energetically active atom to be the center of adsorption is not exposed. As in the case of the interaction between molecules constituting the molecular crystal described above, the adsorption phenomenon caused to occur on the surface of the molecular crystal by interaction with another substance mainly due to van der Waals interaction, hydrogen bonding, or the like.

Generally, the adsorptive property of another molecule to a surface of a molecular crystal as illustrated in FIG. 1A is obtained by estimating the magnitude relationship, tendency, and the like from a molecular structure, such as the type, chain length, and morphology of a functional group of a molecule constituting the molecular crystal or a molecule to be adsorbed. The adsorptive property of the molecular crystal to the surface of the substance as illustrated in FIG. 1B is similarly obtained.

In the present embodiment, a learned model M1 generated in a learning device 1 predicts the interaction index Iᵢₙₜ from a descriptor of a molecular structure of a molecule to be input. The interaction index Iᵢₙₜ correlates with the magnitude of adsorption energy caused by intermolecular bonding (for example, hydrogen bonding) between a molecule and a molecular crystal, and can be used as an index representing the magnitude of the adsorptive property of the molecule to the surface of the molecular crystal. The interaction index Iᵢₙₜ can be directly obtained from an adsorption structure, generated by a molecular simulation or the like, in which a molecular crystal and a molecule are adsorbed to each other. The interaction index Iᵢₙₜ is an index correlating with the magnitude of the adsorption energy as described above. Therefore, by using the interaction index Iᵢₙₜ, the mode of the interaction between the molecular crystal and the molecule and the magnitude of the strength thereof in the adsorption phenomenon between the molecular crystal and the molecule can be predicted quantitatively and visually, and thus the adsorptive property of the molecule adsorbed to the molecular crystal can be predicted and the adsorptive properties of the molecules adsorbed to the molecular crystal can be easily compared.

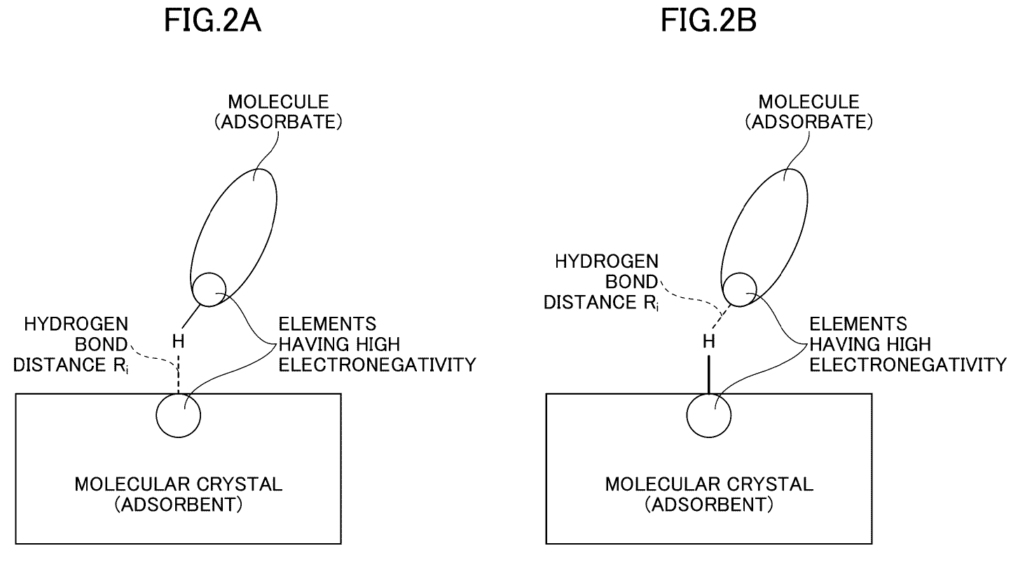

Additionally, the interaction index Iᵢₙₜ correlates with the distance of a molecular bond in adsorption between a molecular crystal and a molecule. The interaction index Iᵢₙₜ can be expressed by a function in which the index decreases as the distance of the molecular bonding in the adsorption increases, and thus can be regarded as an index representing the strength of the hydrogen bonding in the adsorption of the molecule of interest. When a molecule is adsorbed to a surface of a molecular crystal, the distance of the molecular bond may be obtained by determining one atom on the surface of the molecular crystal and one atom of the molecule as representative coordinates. For example, as illustrated in FIG. 2A and FIG. 2B, when the molecular bond is a hydrogen bond, the hydrogen bond distance Rᵢ is obtained as the distance between a hydrogen atom added to an element having a high electronegativity in the adsorbed molecule and an element having a high electronegativity in the molecular crystal (see FIG. 2A). Additionally, the hydrogen bond distance Rᵢ is obtained as the distance between a hydrogen atom added to an element having a high electronegativity in the molecular crystal and an element having a high electronegativity in the adsorbed molecule (see FIG. 2B).

In the present embodiment, the interaction index Iᵢₙₜ output from the learned model M1 can be used as an index of the distance of the molecular bond in the adsorption between the molecular crystal and the molecule, that is, the strength of the molecular bonding. Thus, by outputting the interaction index Iᵢₙₜ from a descriptor of a molecular structure of a molecule input for each molecular crystal, the magnitude of the adsorptive property of the molecule to the surface of the molecular crystal can be understood.

Here, the element having a high electronegativity is not particularly limited, and examples thereof include oxygen, nitrogen, sulfur, fluorine, chlorine, bromine, and iodine.

Additionally, when focusing on the interaction between the π electron of the aromatic ring or the like and another functional group or the like, instead of the distance of the molecular bond in the adsorption, the distance between the centroid of the aromatic ring and another functional group or the like that are used as the representative coordinates may be obtained as the distance of the molecular bond.

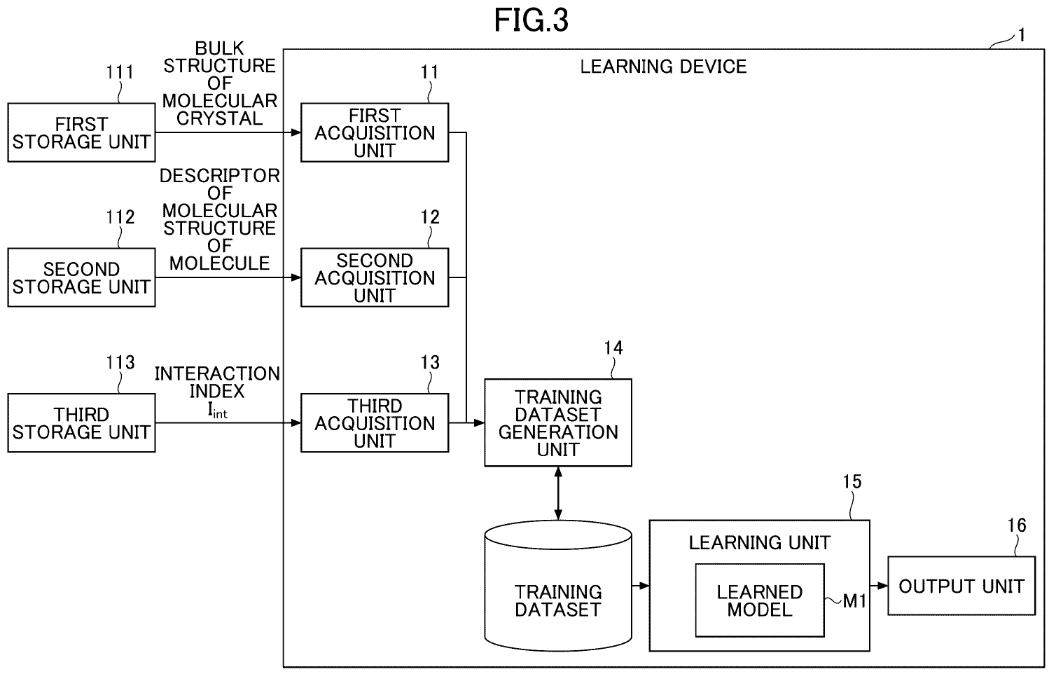

FIG. 3 is a block diagram illustrating a schematic configuration of the learning device according to the present embodiment. As illustrated in FIG. 3, the learning device 1 includes a first acquisition unit 11, a second acquisition unit 12, a third acquisition unit 13, a training dataset generation unit 14, a learning unit 15, and an output unit 16. The learning device 1 generates the learned model M1 that predicts the interaction index Iᵢₙₜ from the descriptor of the molecular structure of the molecular crystal.

The first acquisition unit 11 acquires a bulk structure of the molecular crystal.

Information such as the bulk structure of the molecular crystal may be acquired from a first storage unit 111.

The first storage unit 111 stores a data table including the bulk structure of the molecular crystal and the like. The data table is not particularly limited as long as it is a database including the bulk structure of the molecular crystal and the like, and may be a general database, and for example, a crystallography open database or the like may be used.

The second acquisition unit 12 acquires the descriptor of the molecular structure of the molecule as an explanatory variable.

The information on the descriptor of the molecular structure of the molecule may be acquired from a second storage unit 112.

The second storage unit 112 stores a data table including data related to the molecule, such as a molecular structure of the molecule. The data table is not particularly limited as long as it is a database including data related to the molecule, and may be a general database, and for example, Pubchem, PubChemQC, ZINC, ChemSpider, Chembl, GDB, QM7, QM8, QM9, or the like can be used.

Additionally, the second storage unit 112 may store, for example, RDKit and the like included in a library of Anaconda (registered trademark), which is software distributed from Anaconda Corporation in the United States and the like. When a structural notation is SMILES, the second acquisition unit 12 reads a character string of SMILES by using the MolFromSmiles included in RDKit, and reads the structural notation of the molecule. Here, SMILES is a character string representing a molecular structure of a molecule. The structural notation of each molecule may be recorded in a table described in a data format, such as CSV or Excel, which is spreadsheet software.

SMILES may be obtained, for example, from a chemical database, such as PubChem (the database for chemical substances provided by NCBI in the United States).

The third acquisition unit 13 acquires the interaction index Iᵢₙₜ as an objective variable.

The information on the interaction index Iᵢₙₜ may be acquired from a third storage unit 113.

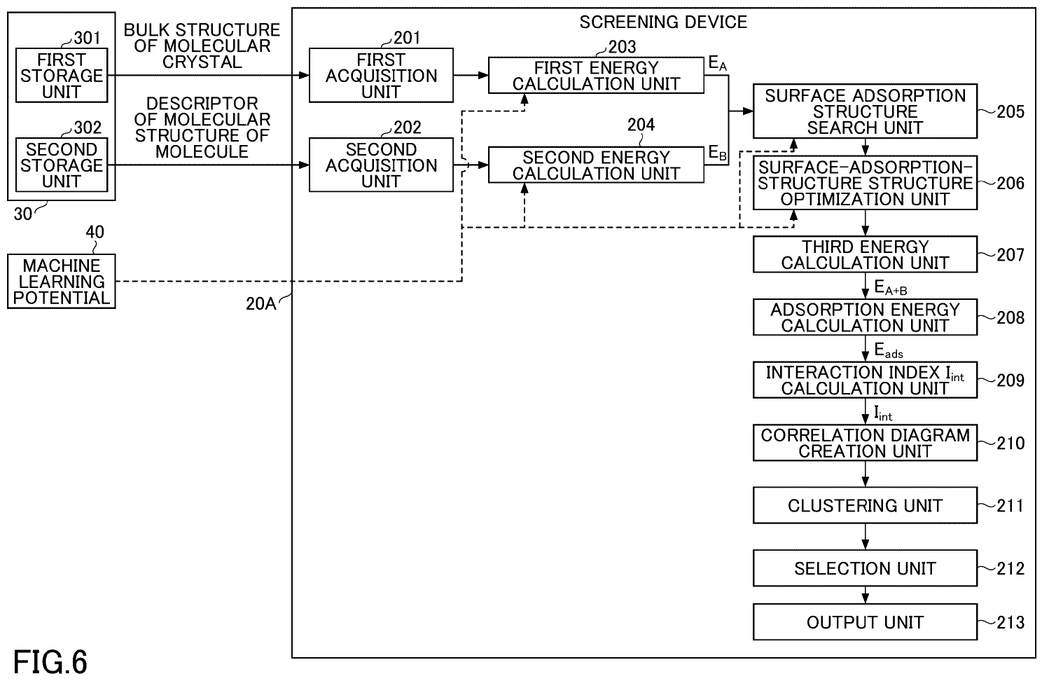

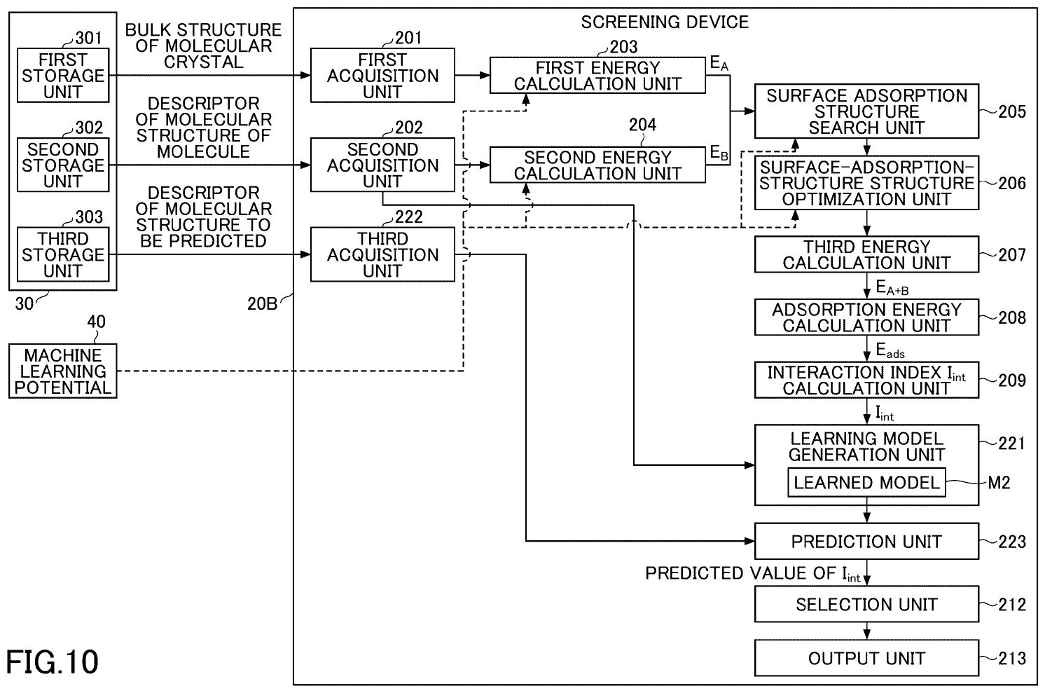

The third storage unit 113 stores a data table including information on the interaction index Iᵢₙₜ and the like. The data table is only required to be a database including information on the interaction index Iᵢₙₜ and the like. The third storage unit 113 may use, for example, a database in which the interaction index Iᵢₙₜ calculated by an interaction index calculation unit 209 of a screening device 20A (see FIG. 6) or a screening device 20B (see FIG. 10) described later is recorded.

The training dataset generation unit 14 extracts the descriptor of the molecular structure of the molecule to be adsorbed as the explanatory variable and the interaction index Iᵢₙₜ as the objective variable, and adds the extracted variables to a training dataset. The training dataset generation unit 14 generates the training dataset by associating the input descriptor of the molecular structure of the molecule with the input interaction index Iᵢₙₜ.

The learning unit 15 generates the learned model M1 by performing learning using the training dataset in which the descriptor of the molecular structure of the molecule (the explanatory variable) is associated with the interaction index Iᵢₙₜ (the objective variable).

The learned model M1 is a learned model on which machine learning is performed in advance by using the training dataset (a training data table) stored in a storage unit, which is not illustrated, and a learning result of a correspondence relationship between the descriptor of the molecular structure of the molecule (the explanatory variable) and the interaction index Iᵢₙₜ (the objective variable), obtained by performing machine learning using the training dataset stored in the storage unit, is applied. The learned model M1 is a program for using the descriptor of the molecular structure of the molecule (the explanatory variable) as input data, and using the interaction index Iᵢₙₜ (the objective variable) as output data to model the input-output relationship between the descriptor of the molecular structure of the molecule and the interaction index Iᵢₙₜ. Here, the learned model M1 may be represented by a mathematical expression, such as a function.

To the learned model M1, a supervised learning algorithm is preferably applied among machine learning algorithms. Examples of the supervised learning include linear regression, regularized regression, partial least squares regression, polynomial regression, kernel regression, logistic regression, random forest, gradient boosting regression tree, a support vector machine (SVM), and a neural network. As the neural network, deep learning in which the neural network is formed in more than three layers can be used. As the type of the neural network, for example, a convolutional neural network (CNN), a recurrent neural network (RNN), a general regression neural network, or the like can be used. Among these, the gradient boosting regression tree is preferably used.

The output unit 16 outputs information on the training dataset used in the learning of the learned model M1, information related to the learned model M1, and the like by displaying or the like.

As described above, the learning device 1 includes the learning unit 15, and thus can generate the learned model M1 that predicts the interaction index Iᵢₙₜ from the descriptor of the molecular structure of the molecule. The learned model M1 generated by the learning device 1 can predict the interaction index Iᵢₙₜ from the input descriptor of the molecular structure of the molecule.

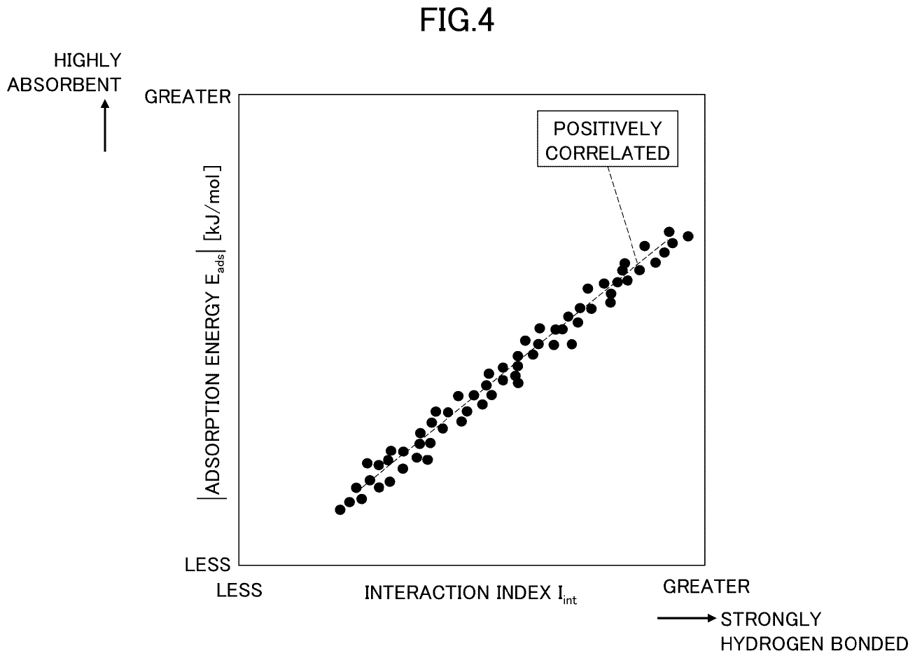

As illustrated in FIG. 4, when the interaction index Iᵢₙₜ increases, the absolute value of the adsorption energy E_{ads} tends to increase, and there is a positive correlation between the interaction index Iᵢₙₜ and the absolute value of the adsorption energy E_{ads}. Thus, predicting the interaction index Iᵢₙₜ by inputting the descriptor of the molecular structure of the molecule into the learned model M1 generated by the learning device 1 is synonymous with checking the adsorption energy of the molecule, and thus the magnitude of the adsorptive property can be predicted. Therefore, the learning device 1 can be used to predict the adsorptive property of the molecule without directly obtaining the adsorption energy of the molecule adsorbed to the surface of the molecular crystal.

Additionally, the learning device 1 can generate the learned model M1, and thus can use the learned model M1 to predict the interaction index Iᵢₙₜ from the descriptor of the molecular structure of the molecule. By using the predicted interaction index Iᵢₙₜ for predicting the magnitude of the adsorptive property of the molecule, the load and time required to select a molecule having a predetermined magnitude of the adsorptive property can be reduced. When selecting a molecule effective for a molecular crystal, a molecule having a high adsorptive property is selected according to the type of the molecular crystal or the like, and therefore, in practice, various molecular crystals and molecules are combined in experiments, and the magnitude of the adsorption energy or the adsorptive property of the molecule is checked. By performing such a process, a great deal of labor is required to select a molecule having an appropriate adsorptive property to the molecular crystal, and the cost is also high due to the preparation of various molecular crystals and molecules. The interaction index Iᵢₙₜ can be predicted from the descriptor of the molecular structure of the molecule by using the learned model M1 generated by the learning device 1, thereby reducing the load in predicting the adsorptive property of the molecule to the molecular crystal, and shortening the time required to select a molecule having a suitable adsorptive property.

### <Screening System>

A screening system according to the embodiment of the present invention will be described. The screening system according to the present embodiment screens molecules by using the interaction index Iᵢₙₜ calculated as the index representing the magnitude of adsorptive property from the descriptor of the molecular structure of the molecule.

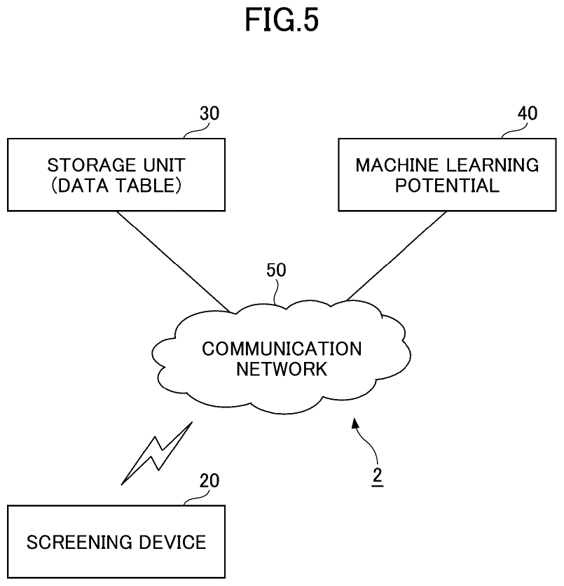

FIG. 5 is a diagram illustrating a configuration of the screening system according to the present embodiment. As illustrated in FIG. 5, a screening system 2 includes a screening device 20, a storage unit 30, and a machine learning potential 40. In the screening system 2, the screening device 20, the storage unit 30, and the machine learning potential 40 may be connected via a communication network 50, and input values to the screening device 20, the storage unit 30, and the machine learning potential 40 and output values of the screening device 20, the storage unit 30, and the machine learning potential 40 may be transmitted via the communication network 50. At least either the storage unit 30 or the machine learning potential 40 may be stored on a cloud.

Here, in the present embodiment, the screening device 20, the storage unit 30, and the machine learning potential 40 are connected via the communication network 50, and may be connected by wire. Additionally, the screening system 2 may be a single device such as a personal computer (PC) including each component inside the device.

The screening device 20 predicts the interaction index Iᵢₙₜ when the adsorbate is adsorbed on the adsorbent by using the machine learning potential 40. Here, in the present embodiment, as in the learning device 1 described above, a case where the adsorbent is a molecular crystal and the adsorbate is a molecule to be adsorbed to the molecular crystal will be described. The screening device 20 will be described in detail later.

The storage unit 30 stores a data table including information on the molecular crystal, the molecule, and the like.

The storage unit 30 includes a first storage unit 301 (see FIG. 6) configured to store the bulk structure of the molecular crystal, a second storage unit 302 (see FIG. 6) configured to store information on the descriptor of the molecular structure of the molecule, and may include a third storage unit 303 (see FIG. 10) configured to store information on a descriptor of a molecular structure of a prediction target molecule.

As the machine learning potential 40, an interatomic potential using a machine learning method that outputs energy from information on a structure of an atom is applied. Examples of the machine learning potential include a neural network potential (NNP), a Gaussian approximation potential (GAP), a spectral neighbor analysis potential (SNAP), and a moment tensor potential (MTP). Among these, NNP is preferable as the machine learning potential in terms of high flexibility of the neural network. NNP can use an atomic simulator that has learned a relationship between coordinates of the atom and energy by using quantum chemical calculation as a training dataset. As NNP, MATLANTIS (trademark) may be used.

### [Screening Device]

The screening device 20 will be described. The screening device 20 screens molecules by using the interaction index Iᵢₙₜ calculated as the index representing the magnitude of the adsorptive property of the molecule from the descriptor of the molecular structure of the molecule. The screening device 20 may be any device that can screen a molecule by using the interaction index Iᵢₙₜ calculated from the descriptor of the molecular structure of the molecule. In describing the screening device 20, two embodiments of the screening device 20 will be described. One embodiment of the screening device 20 is referred to as the screening device 20A (see FIG. 6), and the other embodiment is referred to as the screening device 20B (see FIG. 10).

### First Embodiment

The screening device according to the first embodiment will be described. FIG. 6 is a system configuration diagram illustrating a configuration of the screening device according to the first embodiment. As illustrated in FIG. 6, the screening device 20A according to the first embodiment includes a first acquisition unit 201, a second acquisition unit 202, a first energy calculation unit 203, a second energy calculation unit 204, a surface adsorption structure search unit 205, a surface-adsorption-structure structure optimization unit 206, a third energy calculation unit 207, an adsorption energy calculation unit 208, the interaction index calculation unit 209, a correlation diagram creation unit 210, a clustering unit 211, a selection unit 212, and an output unit 213. The screening device 20A calculates the interaction index Iᵢₙₜ as the index representing the magnitude of the adsorptive property from the descriptor of the molecular structure of the molecule, and select a candidate molecule group.

The screening device 20A may perform processing by using the machine learning potential 40 in any one of the first energy calculation unit 203, the second energy calculation unit 204, the surface adsorption structure search unit 205, the surface-adsorption-structure structure optimization unit 206, the third energy calculation unit 207, the adsorption energy calculation unit 208, the interaction index calculation unit 209, the correlation diagram creation unit 210, the clustering unit 211, or the selection unit 212. The screening device 20A preferably preforms processing by using the machine learning potential 40 in any one or all of the first energy calculation unit 203, the second energy calculation unit 204, the surface adsorption structure search unit 205, and the surface-adsorption-structure structure optimization unit 206, which have a particularly large calculation load.

The first acquisition unit 201 acquires the bulk structure of the molecular crystal. The bulk structure of the molecular crystal may be acquired from the first storage unit 301 of the storage unit 30. The first storage unit 301 stores the bulk structure of the molecular crystal, as with the first storage unit 111 described above. The information on the molecular crystal is substantially the same as the information on the molecular crystal acquired from the first storage unit 111 in the learning device 1 described above, and thus the description thereof will be omitted.

The second acquisition unit 202 acquires the descriptor of the molecular structure of the molecule as the explanatory variable. The information on the descriptor of the molecular structure of the molecule may be acquired from the second storage unit 302 of the storage unit 30. The second storage unit 302 stores the information on the descriptor of the molecular structure of the molecule, as with the second storage unit 112 described above. The information on the descriptor of the molecular structure of the molecule is substantially the same as the information on the descriptor of the molecular structure of the molecule acquired from the second storage unit 112 in the learning device 1 described above, and thus the description thereof will be omitted.

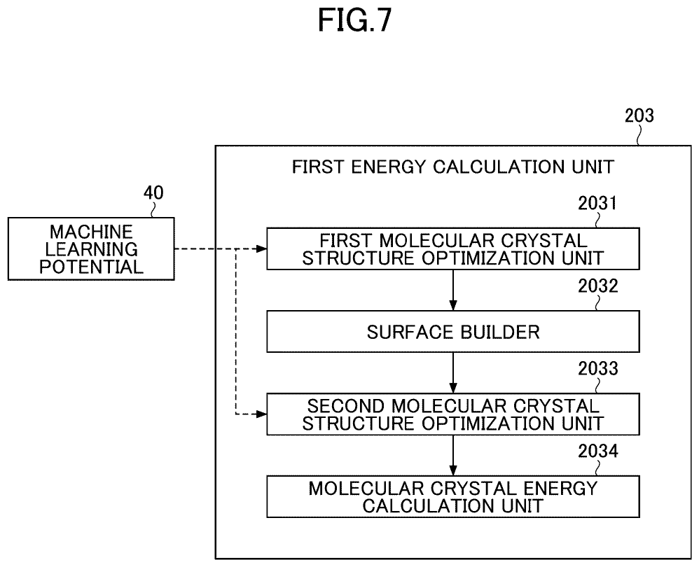

The first energy calculation unit 203 calculates the energy of the bulk structure of the molecular crystal. As illustrated in FIG. 7, the first energy calculation unit 203 may include a first molecular crystal structure optimization unit 2031, a surface builder 2032, a second molecular crystal structure optimization unit 2033, and a molecular crystal energy calculation unit 2034.

The first energy calculation unit 203 preferably performs processing by using the machine learning potential 40 in the first molecular crystal structure optimization unit 2031 and the second molecular crystal structure optimization unit 2033, which have a particularly large calculation load.

The first molecular crystal structure optimization unit 2031 performs structure optimization of the bulk structure of the molecular crystal. The calculation accuracy is improved by performing the structure optimization of the bulk structure.

As a method of optimizing the bulk structure of the molecular crystal, a generally used method may be used.

For example, the first molecular crystal structure optimization unit 2031 may obtain a structure in which the bulk structure of the molecular crystal is energetically most stable by arranging the molecules at appropriate positions in consideration of the relationship between the coordinates and energy of the atoms.

Additionally, the first molecular crystal structure optimization unit 2031 may perform the structure optimization of the bulk structure of the molecular crystal by using a general molecular simulation method, such as first-principles calculation or molecular mechanics calculation.

Further, the first molecular crystal structure optimization unit 2031 may create an optimized bulk structure of the molecular crystal, optimized by relaxing the bulk structure of the molecular crystal. Here, relaxing is a general structure optimization method, such as a steepest descent method or a conjugate gradient method, for obtaining the minimum value of energy in a multi-dimensional space. For example, relaxing indicates performing an operation so that a sum or scalar of force vectors acting on the entire bulk structure of a molecular crystal, or the stress tensor, the main component of the stress tensor of the force vectors, or the like matches with a predetermined pressure (external pressure) under a certain threshold value. The bulk structure of the molecular crystal acquired by the first acquisition unit 201 may have density that is not adapted to the actual state, which is incorrect, because monomolecular information of the bulk structure of the molecular crystal is input into a simulation cell having a given size. In this state, when the energy E_{A} of the bulk structure of the molecular crystal is calculated by the molecular crystal energy calculation unit 2034, the accuracy of the calculated energy E_{A} may be low. The first molecular crystal structure optimization unit 2031 can create the optimized bulk structure of the molecular crystal, adjusted to a bulk structure of the molecular crystal having correct density, by optimizing the bulk structure of the molecular crystal.

When the first molecular crystal structure optimization unit 2031 relaxes the bulk structure of the molecular crystal to create the optimized bulk structure of the molecular crystal, the optimized bulk structure may be created under the following two conditions.
(1) The volume of the simulation cell is variable. An external force may be applied to the simulation cell, and a predetermined pressure (external pressure) may be applied to the bulk structure of the molecular crystal.
(2) The first molecular crystal structure optimization unit 2031 may set the above (1) as a structure at absolute zero. In order to consider the influence of temperature, a molecular dynamics simulation may be performed. In addition to the molecular dynamics simulation, a molecular simulation, such as a Monte Carlo method, may be performed. The volume of the simulation cell may be variable. The temperature may be set to any appropriate value. An external force may be applied to the simulation cell, and a predetermined pressure (external pressure) may be applied to the bulk structure of the molecular crystal.

By creating the optimized bulk structure of the molecular crystal under the above two conditions, the simulation cell is set to reproduce the actual density of the bulk structure of the molecular crystal. That is, the simulation cell can be set to simulate the density when the bulk structure of the molecular crystal is relaxed, in consideration of the predetermined pressure and temperature.

As described above, the first molecular crystal structure optimization unit 2031 preferably performs processing by using the machine learning potential 40 because the calculation load is particularly large in the first energy calculation unit 203. This enables the structure optimization of the bulk structure of the molecular crystal to be performed in a shorter time.

The surface builder 2032 cuts the optimized bulk structure of the molecular crystal to build a surface having a selected plane index.

The selected plane index may be set to any appropriate plane index in accordance with the type of the bulk structure of the molecular crystal.

The second molecular crystal structure optimization unit 2033 performs structure optimization of the bulk structure of the molecular crystal in which the surface of the selected plane index is built. By performing the structure optimization of the bulk structure of the molecular crystal in which the surface of the selected plane index is built, the calculation accuracy of the energy of the bulk structure of the molecular crystal in which the surface of the selected plane index is built is improved.

The second molecular crystal structure optimization unit 2033 may perform the structure optimization in substantially the same manner as the first molecular crystal structure optimization unit 2031. The details of the structure optimization method are substantially the same as those of the first molecular crystal structure optimization unit 2031, and thus the details thereof will be omitted.

As described above, the second molecular crystal structure optimization unit 2033 preferably performs processing by using the machine learning potential 40 because the calculation load is particularly large in the first energy calculation unit 203, as with the first molecular crystal structure optimization unit 2031. This enables the structure optimization of the bulk structure of the molecular crystal, in which the surface of the selected plane index is built, to be performed in a shorter time.

The molecular crystal energy calculation unit 2034 calculates the energy E_{A} of the bulk structure of the molecular crystal in which the surface is built, as first energy.

The energy E_{A} of the bulk structure of the molecular crystal can be calculated using the machine learning potential 40, but the method of calculating the energy E_{A} of the bulk structure of the molecular crystal is not particularly limited, and an empirical potential such as optimized potentials for liquid simulations (OPLS), or the Schrodinger equation, an equation based thereon, and the like may be used.

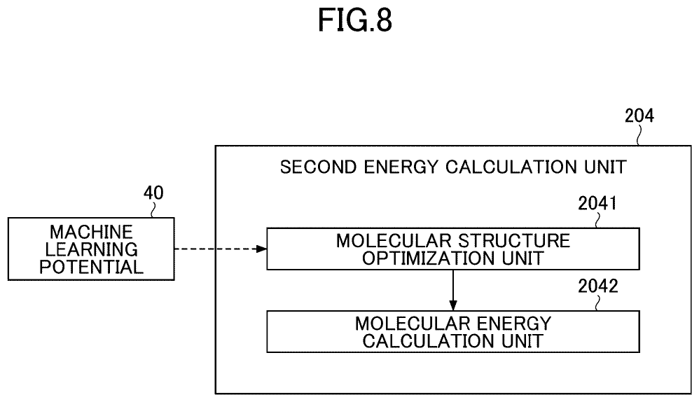

The second energy calculation unit 204 illustrated in FIG. 6 calculates the energy of the molecular structures of multiple (N types (N is an integer of 1 or greater)) molecules. As illustrated in FIG. 8, the second energy calculation unit 204 includes a molecular structure optimization unit 2041 and a molecular energy calculation unit 2042.

The molecular structure optimization unit 2041 performs structure optimization of the molecular structures of multiple molecules. By performing structure optimization of the molecular structure of the molecule, the calculation accuracy of the energy is improved.

The molecular structure optimization unit 2041 may perform the structure optimization by substantially the same method as the first molecular crystal structure optimization unit 2031. The details of the structure optimization method are substantially the same as those of the first molecular crystal structure optimization unit 2031, and thus the details thereof will be omitted.

The molecular structure optimization unit 2041 preferably performs processing by using the machine learning potential 40 because the calculation load is particularly large in the second energy calculation unit 204. This enables the structural optimization of the molecular structure of the molecule to be performed in a shorter time.

The molecular energy calculation unit 2042 calculates the energy E_{B} of the structure-optimized molecule as second energy.

The method of calculating the energy E_{B} of the structure-optimized molecule is not particularly limited, and a general calculation method may be used. For example, a method substantially the same as the method of calculating the energy E_{A} of the bulk structure of the molecular crystal in the molecular crystal energy calculation unit 2034 may be used.

As illustrated in FIG. 6, the surface adsorption structure search unit 205 searches for a suitable surface adsorption structure in structures obtained by combining the bulk structure of the molecular crystal, in which the surface has been built, with the optimized molecular structures of the molecules. The surface adsorption structure may be searched for, for each of the multiple (N types of) optimized molecular structures of the molecules in the molecular structure optimization unit 2041.

The search method is not particularly limited, and a general search method may be used, and for example, Bayes optimization, sequential optimization, random search, grid search, or the like may be used.

The surface-adsorption-structure structure optimization unit 206 performs structure optimization of the surface adsorption structure.

The surface-adsorption-structure structure optimization unit 206 may perform the structure optimization by substantially the same method as the first molecular crystal structure optimization unit 2031. The details of the structure optimization method are substantially the same as those of the first molecular crystal structure optimization unit 2031, and thus the details thereof will be omitted.

The third energy calculation unit 207 calculates the energy E_{A+B} of the structure-optimized surface adsorption structure as third energy.

The method of calculating the energy E_{A}+B of the structure-optimized surface adsorption structure is not particularly limited, and a general calculation method may be used. For example, a method substantially the same as the method of calculating the energy E_{A} of the bulk structure of the molecular crystal in the molecular crystal energy calculation unit 2034 may be used.

The adsorption energy calculation unit 208 calculates the adsorption energy E_{ads} of the molecule in the structure-optimized surface adsorption structure.

The adsorption energy E_{ads} of the molecule in the structure-optimized surface adsorption structure can be calculated from the following equation.

$${E}_{ads} = {E}_{A + B} - ( {E}_{A} + {E}_{B} ) \qquad ( I )$$

The interaction index calculation unit 209 calculates the interaction index Iᵢₙₜ of the structure-optimized surface adsorption structure.

The interaction index Iᵢₙₜ is only required to be a value calculated as an index representing the interaction of the intermolecular bond of interest between the molecule as the adsorbate and the molecular crystal as the adsorbent, and for example, a value calculated using the above equation (1) can be used.

The correlation diagram creation unit 210 creates a relationship diagram indicating the correlation between the interaction index Iᵢₙₜ of the intermolecular bond in the structure-optimized surface adsorption structure and the absolute value of the adsorption energy E_{ads} of the molecule in the surface adsorption structure.

The clustering unit 211 performs clustering on the adsorption structures of the surface-optimized surface adsorption structures based on the distance between the molecular crystal and the molecule, and extracts multiple candidate molecule groups. The clustering unit 211 may extract and classify only a predetermined number (for example, 100) of molecules, based on the distance between the molecular crystal and the molecule in the created surface adsorption structure, in the order from the shortest in the distance between the molecular crystal and the molecule. Additionally, the clustering unit 211 may extract only a predetermined number (for example, 100) of molecules, based on the interaction index Iᵢₙₜ of the intermolecular bond in the structure-optimized surface adsorption structure or the absolute value of the adsorption energy E_{ads} of the molecule in the surface adsorption structure, in the order from the largest in the interaction index Iᵢₙₜ or the absolute value of the adsorption energy E_{ads} of the molecule.

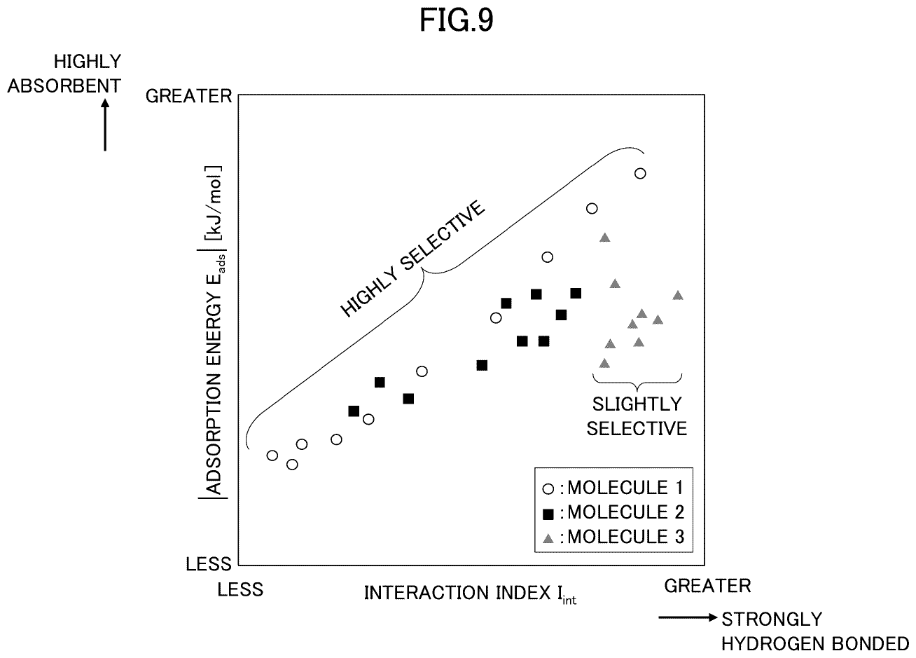

By performing the clustering to extract the candidate molecule groups, the degree of selectivity of the molecular crystal for the molecules can be found from the degree of dispersion in the candidate molecule groups. For example, as illustrated in FIG. 9, molecule groups are extracted for each of three types of molecules: molecule 1, molecule 2, and molecule 3. It can be found that for molecule 1, the selectivity for a certain adsorbent is high because the values of the interaction index Iᵢₙₜ are distributed over a wide range, and can be found that for molecule 3, the selectivity for a certain adsorbent is low, because the values of the interaction index Iᵢₙₜ are distributed over only a narrow range.

As the clustering method, a generally used method can be used, and as the clustering method, for example, the k-Means method, the k-Means++ method, or the Gaussian Mixture method is used. The k-Means method is a method of classifying molecules into k clusters. For example, when the k-Means method is used, a vector x in which values of variables of predetermined molecules are stored is randomly assigned to k clusters. Next, the centroid of the molecules assigned to each cluster is calculated. Next, for each molecule, the distance from the calculated centroid is calculated, and the vector x is reassigned to the clusters with the shortest distance. Until the assignment of all the molecules to the clusters converges, the process of calculating the centroid of the molecules assigned to each of the clusters and the process of calculating the distances from the calculated centroid for each of the molecules and reassigning the vector x to the clusters with the shortest distance are repeated.

Additionally, the clustering unit 211 may perform the clustering after visualizing the extracted candidate molecule groups and performing nonlinear dimension reduction of high-dimensional data to two dimensions or three dimensions. By calculating the distances between the molecular crystal and the molecules in the surface adsorption structures of the extracted candidate molecule groups, variables of the dimensions, of which the number corresponding to the number of extracted molecule groups (for example, 100 dimensions when 100 molecule groups are extracted) are obtained. The clustering unit 211 visualizes the extracted candidate molecule groups and performs nonlinear dimensionality reduction of the high-dimensional data to two dimensions or three dimensions, thereby facilitating appropriate clustering.

Examples of the method of visualizing the extracted candidate molecule groups include principal component analysis (PCA) and the like. The clustering unit 211 projects data of the extracted candidate molecule groups, that is obtained by performing rotational transformation of the coordinate system centered at the sample average by using PCA, to a lower-dimensional space, and thus can visualize the data of the extracted candidate molecule groups so that the scatter of points appears as large as possible with a smaller number of coordinate axes.

As a method of performing the nonlinearly reduction on the high-dimensional data to two dimensions or three dimensions, for example, a t-distributed stochastic neighbor embedding (t-SNE) method of maintaining a relationship of the intermolecular distance between the molecular crystal and the molecule, generative topographic mapping (GTM) of maintaining a positional relationship between the molecular crystal and the molecule, or the like is used.

The selection unit 212 selects a candidate molecule group from among the multiple molecules. The selection unit 212 may select a suitable molecule group as the candidate molecule group, depending on the magnitude of the adsorptive property required for the molecule.

The method of selecting the molecule group is not particularly limited, and a general selection method may be used. As the selection method, the selection unit 212 may select the molecule group based on, for example, a condition that a predetermined number of molecules in the order from the largest in the interaction index Iᵢₙₜ or the absolute value of the adsorption energy E_{ads} are included, a condition that the interaction index Iᵢₙₜ or the absolute value of the adsorption energy E_{ads} is greater than or equal to a predetermined value, a condition that the interaction index Iᵢₙₜ or the absolute value of the adsorption energy E_{ads} is the highest, or the like.

The output unit 213 outputs the candidate molecule group by displaying or the like.

The screening device 20A includes the first energy calculation unit 203, the second energy calculation unit 204, the surface adsorption structure search unit 205, the surface-adsorption-structure structure optimization unit 206, the third energy calculation unit 207, the adsorption energy calculation unit 208, the interaction index calculation unit 209, the correlation diagram creation unit 210, the clustering unit 211, and the selection unit 212. The screening device 20A can select multiple molecules as multiple molecule groups according to the adsorption strength of molecules adsorbed to the molecular crystal by performing clustering based on the interaction index Iᵢₙₜ and the absolute values of the adsorption energy E_{ads} in the clustering unit 211. With this, the screening device 20A can extract a candidate molecule group having a suitable adsorption magnitude from among the multiple molecules. The adsorptive property between the molecular crystal and the molecule in the surface adsorption structure can be found from the interaction index Iᵢₙₜ and the absolute value of the adsorption energy E_{ads}. Therefore, the screening device 20A can predict the adsorptive property of the molecule without directly obtaining the adsorption energy of the molecule adsorbed to the surface of the molecular crystal, and thus can extract a candidate molecule group having a suitable adsorption magnitude.

### Second Embodiment

The screening device according to the second embodiment will be described. FIG. 10 is a system configuration diagram illustrating a configuration of the screening device according to the second embodiment. As illustrated in FIG. 10, the screening device 20B includes a learning model generation unit 221, a third acquisition unit 222, and a prediction unit 223 instead of the correlation diagram creation unit 210 and the clustering unit 211 in the screening device 20A illustrated in FIG. 6, and predict the interaction index Iᵢₙₜ as the index representing the magnitude of the adsorptive property from the descriptor of the molecular structure of the prediction target molecule.

The learning model generation unit 221 generates a learned model M2 by using the descriptor of the molecular structure of the molecule (an explanatory variable) acquired by the second acquisition unit 202 and the interaction index Iᵢₙₜ (an objective variable) calculated by the interaction index calculation unit 209.

That is, the learning model generation unit 221 can generate the learned model M2 by creating a training dataset in which the descriptor of the molecular structure of the molecule (the explanatory variable) acquired by the second acquisition unit 202 is associated with the interaction index Iᵢₙₜ (the objective variable) calculated by the interaction index calculation unit 209, and performing learning using the created training dataset.

As the learned model M2, the learned model M1 generated by the learning device 1 can be used.

The third acquisition unit 222 acquires, as the explanatory variable, the descriptor of the molecular structure of the prediction target molecule. The information on the descriptor of the molecular structure of the prediction target molecule may be acquired from the third storage unit 303 of the storage unit 30. The third storage unit 303 stores information of the descriptor of the molecular structure of the molecule, as with the second storage unit 112 described above. The information on the descriptor of the molecular structure of the molecule is substantially the same as the information on the descriptor of the molecular structure of the molecule acquired from the second storage unit 112 in the learning device 1 described above, and thus the description thereof will be omitted.

The prediction unit 223 predicts the interaction index Iᵢₙₜ by inputting the descriptor of the molecular structure of the prediction target molecule, acquired by the third acquisition unit 222, by using the learned model M2.

That is, the prediction unit 223 inputs the descriptor of the molecular structure of the prediction target molecule, acquired by the third acquisition unit 222, into the learned model M2, to output the interaction index Iᵢₙₜ to be predicted, predicted by using the learned model M2, as the objective variable. As described above, the interaction index Iᵢₙₜ correlates with the adsorption energy of the molecule, and thus serves as an index representing the adsorption energy. Therefore, the prediction unit 223 can predict the adsorption energy of the prediction target molecule to the molecular crystal by predicting the interaction index Iᵢₙₜ corresponding to the descriptor of the molecular structure of the prediction target molecule from the learned model M2.

The selection unit 212 selects a candidate molecule from among the multiple molecules. The selection unit 212 may select any multiple molecules as the candidate molecules in accordance with the magnitude of the adsorptive property required for the molecule and the like.

The selection method of selecting the molecule is not particularly limited, and a general selection method may be used. As the selection method, the selection unit 212 may select the molecule based on, for example, a condition that a predetermined number of molecules in the order from the largest in the interaction index Iᵢₙₜ or the absolute value of the adsorption energy E_{ads} are included, a condition that the interaction index Iᵢₙₜ or the absolute value of the adsorption energy E_{ads} is greater than or equal to a predetermined value, or a condition that the interaction index Iᵢₙₜ or the absolute value of the adsorption energy E_{ads} is the largest.

The output unit 213 outputs the selected multiple molecules as the candidate molecules by displaying or the like.

As described above, the screening system 2 includes the screening device 20B, and the screening device 20B includes the learning model generation unit 221, the prediction unit 223, and the selection unit 212. The screening device 20B can predict the interaction index Iᵢₙₜ of the prediction target molecule as the index representing the magnitude of the adsorptive property from the descriptor of the molecular structure of the prediction target molecule by using the learned model M2 in the prediction unit 223. The screening device 20B can predict the adsorptive property of the molecule without performing molecular simulation for obtaining the adsorption structure by predicting the interaction index Iᵢₙₜ of the prediction target. Then, the screening device 20B can select the candidate molecule from among the multiple molecules based on the interaction index Iᵢₙₜ of the prediction target molecule in the selection unit 212. Therefore, the screening device 20B can predict the adsorptive property of the molecule without directly obtaining the adsorption energy of the molecule adsorbed to the surface of the molecular crystal in the prediction unit 223, and thus can select the candidate molecule from among the multiple molecules.

Additionally, in the screening system 2, the screening device 20B can predict the interaction index Iᵢₙₜ of the prediction target molecule by using the learned model M2 in the prediction unit 223. The screening device 20B can predict the magnitude of the adsorptive property of the molecule to the molecular crystal easily and in a shorter time by using the interaction index Iᵢₙₜ predicted by using the learned model M2 in the prediction unit 223, thereby shortening the time required to screen a molecule such as molecules having a high adsorptive property to the molecular crystal and the like.

For example, the screening system 2 can exhaustively screen, select, or design a new substance that has a strong hydrogen bond on the surface of a specific molecular crystal from among a large number of compound groups. Therefore, the screening system 2 can easily compare the adsorptive properties to the molecular crystal between different molecules, thereby performing molecular screening easily and in a shorter time.

Furthermore, in the screening system 2, the screening device 20B can perform processing by using the machine learning potential 40 in at least one of the first energy calculation unit 203, the second energy calculation unit 204, the surface adsorption structure search unit 205, or the surface-adsorption-structure structure optimization unit 206. With this, the screening device 20B can shorten the process time required to obtain an accurate adsorption structure between each molecule and the molecular crystal, for example, when it is desired to find a molecule exhibiting a strong adsorption state by hydrogen bonding from among a large amount of compound groups. Therefore, the screening device 20B according to the present embodiment can shorten the screening time and can also cope with a wider range of molecule groups by enhancing versatility.

### (Hardware Configuration of Learning Device 1 and Screening Device 20 (20A and 20B))

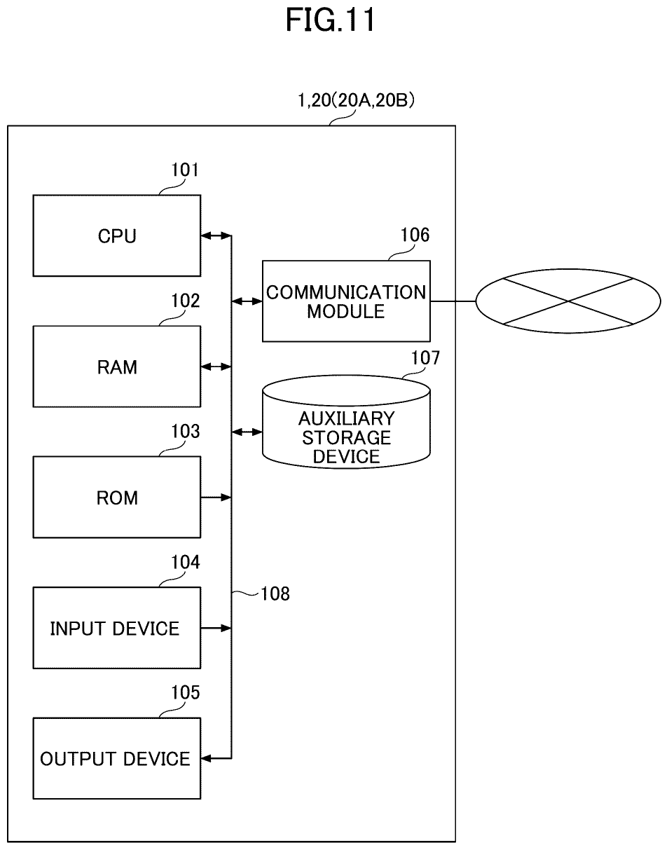

Next, an example of a hardware configuration of the learning device 1 and the screening device 20 will be described. FIG. 11 is a block diagram illustrating the hardware configuration of the learning device 1 and the screening device 20. As illustrated in FIG. 11, the learning device 1 and the screening device 20 are configured as an information processing device (computer), and can be physically configured as a computer system including a central processing unit (CPU: processor) 101 as an arithmetic processing unit, a random access memory (RAM) 102 and a read only memory (ROM) 103 as main storage devices, an input device 104 as an input device, an output device 105, a communication module 106, an auxiliary storage device 107 such as a hard disk drive, and the like. These are connected to each other by a bus 108. Here, the output device 105 and the auxiliary storage device 107 may be provided outside.

The CPU 101 controls the overall operation of the learning device 1 and the screening device 20, and performs various types of information processing. The CPU 101 can generate the learned model M1 or screen a molecule by executing, for example, a learning program or a screening program, which will be described later, stored in the ROM 103 or the auxiliary storage device 107.

The RAM 102 may include a non-volatile RAM that is used as a work area of the CPU 101 and stores main control parameters and information.

The ROM 103 stores a basic input/output program and the like. The learning program and the screening program may be stored in the ROM 103.

The input device 104 is an input device, such as a keyboard, a mouse, an operation button, a touch panel, and a display screen, and receives information input by a user as an instruction signal and outputs the instruction signal to the CPU 101.

The output device 105 is a display device, such as a monitor display or the like, an audio device, such as a speaker or the like, or a printing device, such as a printer or the like. In the output device 105, for example, information such as a selection result of a catalyst and the like is displayed on the display device such as a monitor display or the like, and a screen to be displayed is updated in response to an input operation via the input device 104 or the communication module 106.

The communication module 106 is a data transmission/reception device, such as a network card or the like, and functions as a communication interface that acquires information from an external data recording server or the like and outputs analysis information to another electronic device.

The auxiliary storage device 107 is a storage device, such as a solid state drive (SSD), a hard disk drive (HDD), or the like, and stores, for example, various data and files necessary for the operations of the learning device 1 and the screening devices 20A and 20B, and the like.

The functions of the learning device 1 and the screening device 20 are realized by reading and writing data in the main storage device, such as the RAM 102, or the auxiliary storage device 107 and operating the input device 104, the output device 105, and the communication module 106, by reading predetermined computer software (including the learning program or the screening program) from the main storage device, such as the RAM 102 or the auxiliary storage device 107 and the CPU 101 executing the software.

Thus, the respective units of the learning device 1 and the screening device 20 are realized by cooperation of software and hardware by the processor executing the predetermined computer software (including the learning program or the screening program) stored in advance in a computer including the learning device 1 and the screening device 20.

The learning program and the screening program can be stored in, for example, the main storage device or the auxiliary storage device 107 included in the computer. Additionally, the learning program or the screening program may be stored in a computer connected to a communication line such as the Internet, and a part or all of the learning program or the screening program may be provided by being downloaded via the communication line. Further, the learning program and the screening program may be provided or distributed via the communication line.

The learning program and the screening program may be recorded (including installation) in the computer from a state in which a part or all of the learning program and the screening program are stored in a portable storage medium, such as an optical disk such as a CD-ROM and a DVD-ROM, a semiconductor memory, such as a flash memory, or the like.

### <Learning Method>

A learning method according to the present embodiment will be described. The learning method according to the present embodiment is a method of generating a learned model configured to predict the interaction index Iᵢₙₜ from a descriptor of a molecular structure of a molecule by using a training dataset in which a descriptor of a molecular structure of a molecule is associated with the interaction index Iᵢₙₜ, in the learning device 1 having a configuration as illustrated in FIG. 3.

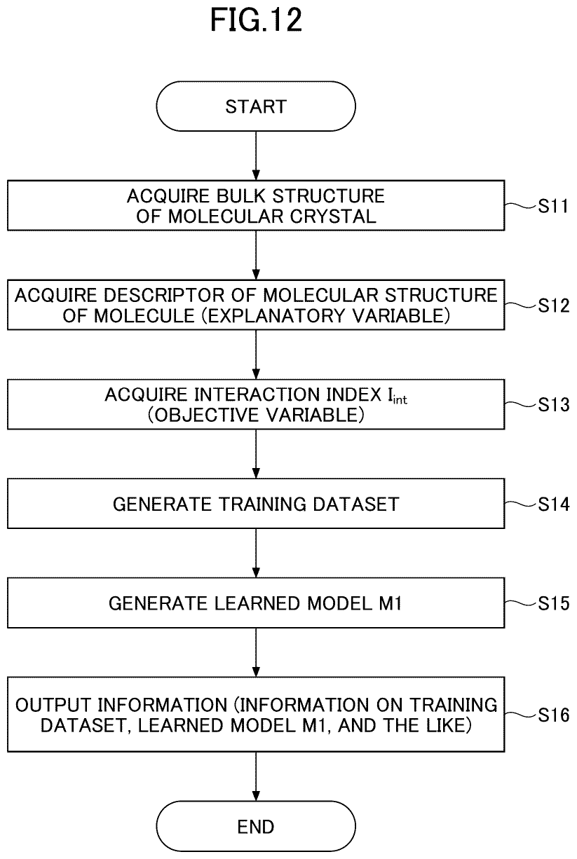

FIG. 12 is a flowchart illustrating the learning method according to the present embodiment. As illustrated in FIG. 12, in the learning method according to the present embodiment, the first acquisition unit 11 acquires the bulk structure of the molecular crystal (a first acquisition step: step S11).

The information on the bulk structure of the molecular crystal and the like may be acquired from the first storage unit 111.

Next, the second acquisition unit 12 acquires the descriptor of the molecular structure of the molecule as the explanatory variable (a second acquisition step: step S12).

The information on the descriptor of the molecular structure of the molecule may be acquired from the second storage unit 112.

Next, the third acquisition unit 13 acquires the interaction index Iᵢₙₜ as the objective variable (a third acquisition step: step S13).

The information on the interaction index Iᵢₙₜ may be acquired from the third storage unit 113.

Next, the training dataset generation unit 14 extracts the descriptor of the molecular structure of the molecule to be adsorbed, as the explanatory variable, and the interaction index Iᵢₙₜ, as the objective variable, and adds the extracted variables to the training dataset (a training dataset creation step: step S14).

The training dataset generation unit 14 associates the input descriptor of the molecular structure of the molecule with the input interaction index Iᵢₙₜ to generate the training dataset.

Next, the learning unit 15 generates the learned model M1 by performing learning using the training dataset in which the descriptor of the molecular structure of the molecule (the explanatory variable) is associated with the interaction index Iᵢₙₜ (the objective variable) (a learned model M1 generation step: step S15).

The learning unit 15 generates the learned model M1 so that the interaction index Iᵢₙₜ is output in accordance with the input descriptor of the molecular structure of the molecule.

Next, the output unit 16 outputs the information on the training dataset used in the learning of the learned model M1, the information related to the learned model M1, and the like, by displaying or the like (an output step: step S16).

The learning method according to the present embodiment includes the learning step (step S15), and in the learning step (step S15), the learned model M1 configured to predict the interaction index Iᵢₙₜ from the descriptor of the molecular structure of the molecule can be generated. The learning method according to the present embodiment can predict the interaction index Iᵢₙₜ from the input descriptor of the molecular structure of the molecule, by using the learned model M1 generated in the learning step (step S15). The interaction index Iᵢₙₜ has a positive correlation with the absolute value of the adsorption energy E_{ads}, and thus can be used as the index representing the magnitude of the adsorption energy. Thus, the learning method according to the present embodiment can predict the adsorption energy of the molecule and predict the magnitude of the adsorptive property by inputting the descriptor of the molecular structure of the molecule into the learned model M1 and predicting the interaction index Iᵢₙₜ. Therefore, the learning method according to the present embodiment can be used to predict the adsorptive property of the molecule without directly obtaining the adsorption energy of the molecule adsorbed to the surface of the molecular crystal.

Additionally, in the learning method according to the present embodiment, the learned model M1 generated in the learning process (step S15) can be used to predict the interaction index Iᵢₙₜ from the descriptor of the molecular structure of the molecule. By using the predicted interaction index Iᵢₙₜ for predicting the magnitude of the adsorptive property of the molecule, the load and time required to predict a molecule having a predetermined magnitude of the adsorptive property can be reduced. The learning method according to the present embodiment can predict the interaction index Iᵢₙₜ from the descriptor of the molecular structure of the molecule by using the generated learned model M1, and thus can reduce the load when predicting the adsorptive property of the molecule to the molecular crystal and can shorten the time required to select a molecule having a suitable adsorptive property by using the predicted interaction index Iᵢₙₜ.

### <Screening Method>

Next, a screening method according to the present embodiment will be described. The screening method according to the present embodiment is a method of screening molecules by using the interaction index Iᵢₙₜ calculated as the index representing the magnitude of the adsorptive property of the molecule from the descriptor of the molecular structure of the molecule.

The screening method according to the present embodiment will be described, as a screening method using the screening device 20A according to the first embodiment above being a screening method according to the first embodiment, and as a screening method using the screening device 20B according to the second embodiment above being a screening method according to the second embodiment.

### First Embodiment

The screening method according to the first embodiment will be described. The screening method according to the first embodiment is a method using the screening device 20A having a configuration as illustrated in FIG. 6. In the screening method according to the first embodiment, the adsorption energy E_{ads} and the interaction index Iᵢₙₜ of the molecule are calculated from the descriptor of the molecular structure of the molecule in the screening device 20A having the configuration as illustrated in FIG. 6, and a candidate molecule group is selected. The screening method according to the first embodiment is performed using the screening device 20A described above, and thus a part of the contents already described will be omitted.

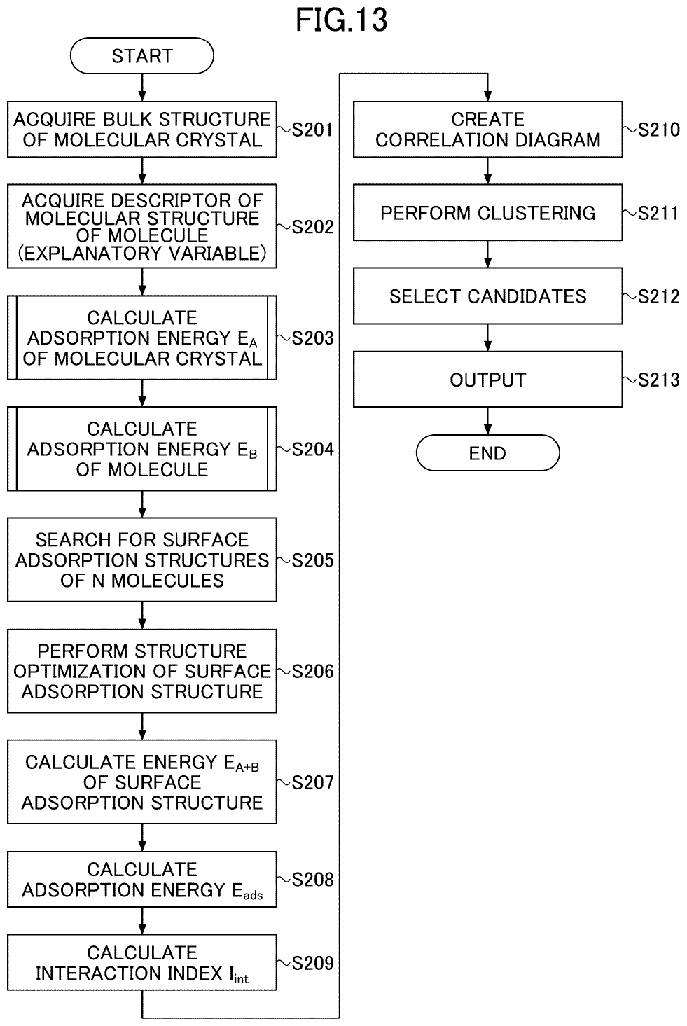

FIG. 13 is a flowchart illustrating the screening method according to the first embodiment. As illustrated in FIG. 13, in the screening method, the first acquisition unit 201 acquires the bulk structure of the molecular crystal (a first acquisition step: step S201).

The bulk structure of the molecular crystal may be acquired from the first storage unit 301 of the storage unit 30.

Next, the second acquisition unit 202 acquires the descriptor of the molecular structure of the molecule as the explanatory variable (a second acquisition step: step S202).

The information on the descriptor of the molecular structure of the molecule may be acquired from the second storage unit 302 of the storage unit 30.

Next, the first energy calculation unit 203 calculates the energy of the bulk structure of the molecular crystal (a first energy calculation step: step S203).

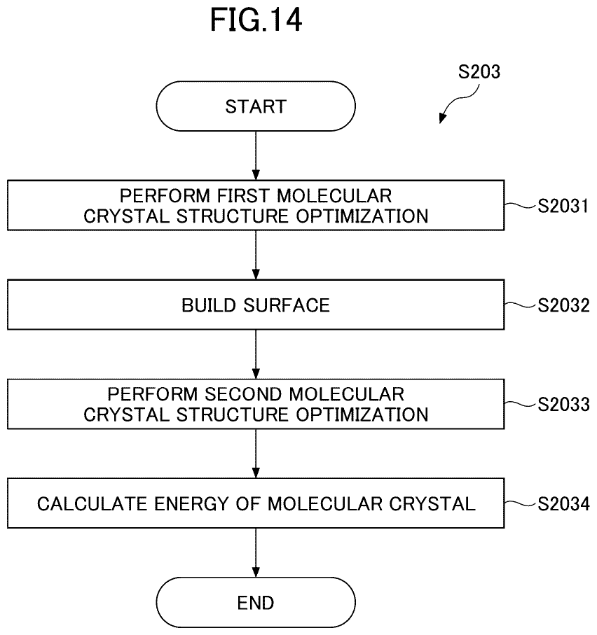

In the first energy calculation step (step S203), as illustrated in FIG. 14, the bulk structure of the molecular crystal is optimized by the first molecular crystal structure optimization unit 2031 (a first molecular crystal structure optimization step: step S2031). By optimizing the bulk structure, the calculation accuracy is improved.

As a method of optimizing the bulk structure of the molecular crystal, a generally used method may be used.

The first molecular crystal structure optimization step (step S2031) has a particularly large calculation load in the first energy calculating process (step S203), and thus is preferably performed by the first molecular crystal structure optimization unit 2031 using the machine learning potential 40. This enables the structure optimization of the bulk structure of the molecular crystal to be performed in a shorter time.

Next, the surface builder 2032 cuts the optimized bulk structure of the molecular crystal to build a surface having a selected plane index (a surface building step: step S2032).

The selected plane index may be set to any appropriate plane index in accordance with the type of the bulk structure of the molecular crystal.

Next, the second molecular crystal structure optimization unit 2033 performs the structure optimization of the bulk structure of the molecular crystal in which the surface of the selected plane index is built (a second molecular crystal structure optimization step: step S2033).

By optimizing the bulk structure of the molecular crystal in which the surface is built, the calculation accuracy of the energy of the bulk structure of the molecular crystal in which the surface is built is improved.

In the second molecular crystal structure optimization step (step S2033), the structure optimization may be performed by the second molecular crystal structure optimization unit 2033 by a method substantially the same as the first molecular crystal structure optimization step (step S2031).

The second molecular crystal structure optimization step (step S2033) has a particularly large calculation load in the first energy calculation step (step S2031), as with the first molecular crystal structure optimization step (step S203), and thus, is preferably performed by the second molecular crystal structure optimization unit 2033 using the machine learning potential 40. This enables the structure optimization of the bulk structure of the molecular crystal, obtained by building the surface of the selected plane index, to be performed in a shorter time.

Next, the molecular crystal energy calculation unit 2034 calculates the energy E_{A} of the bulk structure of the molecular crystal in which the surface is built as the first energy (a molecular crystal energy calculation step: step S2034).

The energy E_{A} of the bulk structure of the molecular crystal can be calculated using the machine learning potential 40, but the method of calculating the energy E_{A} of the bulk structure of the molecular crystal is not particularly limited, and an empirical potential, such as OPLS, or the Schrodinger equation and an equation based thereon may be used.

Next, as illustrated in FIG. 13, the second energy calculation unit 204 calculates the energies of the molecular structures of multiple (N types of (N is an integer of 1 or greater)) molecules (a second energy calculation step: step S204).

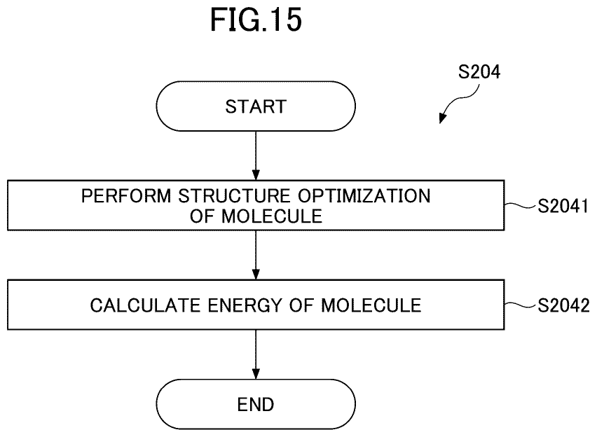

In the second energy calculation step (step S204), as illustrated in FIG. 15, the molecular structure optimization unit 2041 performs structure optimization of the molecular structures of multiple molecules (a molecular structure optimization step: step S2041). By performing the structure optimization of the molecular structure of the molecule, the calculation accuracy of the energy is improved.

In the molecular structure optimization step (step S2041), the molecular structure optimization unit 2041 may perform the structure optimization in substantially the same manner as in the first molecular crystal structure optimization step (step S2031).

The molecular structure optimization step (step S2041) has a particularly large calculation load in the second energy calculating step (step S204), and thus is preferably performed by the molecular structure optimization unit 2041 using the machine learning potential 40. This enables the structural optimization of the molecular structure of the molecule to be performed in a shorter time.

Next, the molecular energy calculation unit 2042 calculates the energy E_{B} of the molecular structure of the structure-optimized molecule as the second energy (a molecular energy calculation step: step S2042).

The calculation method of calculating the energy E_{B} of the structure-optimized molecule is not particularly limited, and a general calculation method may be used. For example, a method substantially the same as the calculation method of calculating the energy E_{A} of the bulk structure of the molecular crystal in the energy calculation step (step S2034) of the molecular crystal may be used.

Next, as illustrated in FIG. 13, the surface adsorption structure search unit 205 searches for a suitable surface adsorption structure in structures obtained by combining the bulk structure of the molecular crystal, in which the surface is built, with the optimized molecular structures of the molecules (a surface adsorption structure search step: step S205).

The surface adsorption structure may be searched for, for each of the molecular optimized structures of multiple (N types of) molecules in a molecular structure optimization step (step S2041).

The search method is not particularly limited, and a general search method may be used, and for example, Bayes optimization, sequential optimization, random search, grid search, or the like may be used.

Next, the surface-adsorption-structure structure optimization unit 206 performs the structure optimization of the surface adsorption structure (a surface-adsorption-structure structure optimization step: step S206).

In the surface-adsorption-structure structure optimization step (step S206), the surface-adsorption-structure structure optimization unit 206 may perform the structure optimization in substantially the same manner as in the first molecular crystal structure optimization step (step S2031).

Next, the third energy calculation unit 207 calculates the energy E_{A+B} of the structure-optimized surface adsorption structure as the third energy (a third energy calculation step: step S207).

The method of calculating the energy E_{A+B} of the structure-optimized surface adsorption structure is not particularly limited, and a general calculation method may be used. For example, a method substantially the same as the method of calculating the energy E_{A} of the bulk structure of the molecular crystal in the energy calculation step (step S2034) of the molecular crystal may be used.

Next, the adsorption energy calculation unit 208 calculates the adsorption energy E_{ads} of the molecule in the structure-optimized surface adsorption structure (an adsorption energy calculation step: step S208).

The adsorption energy E_{ads} of the molecule in the structure-optimized surface adsorption structure can be calculated from the following equation.

$${E}_{ads} = {E}_{A + B} - ( {E}_{A} + {E}_{B} ) \qquad ( I )$$

Next, the interaction index calculation unit 209 calculates the interaction index Iᵢₙₜ of the structure-optimized surface adsorption structure (an interaction index calculation step: step S209).

The interaction index Iᵢₙₜ is only required to be a value calculated as the index representing the interaction of the intermolecular bond of interest between the molecule as the adsorbate and the molecular crystal as the adsorbent, and for example, a value calculated using the above equation (1) can be used.

Next, the correlation diagram creation unit 210 creates a correlation diagram indicating a correlation between the interaction index Iᵢₙₜ of the intermolecular bond in the structure-optimized surface adsorption structure and the absolute value of the adsorption energy E_{ads} of the molecule in the structure-optimized surface adsorption structure (a correlation diagram creation step: step S210).

Next, the clustering unit 211 performs clustering on the adsorption structures of the structure-optimized surface adsorption structures, based on the distance between the molecular crystal and the molecule, and extracts multiple candidate molecule groups (a clustering step: step S211).

In the clustering step (step S211), the clustering unit 211 may extract and classify only a predetermined number of (for example, 100) molecules, based on the distance between the molecular crystal and the molecule in the created surface adsorption structure, from the shortest in the distance between the molecular crystal and the molecule. Additionally, the clustering unit 211 may extract a predetermined number (for example, 100) of molecules, based on the interaction index Iᵢₙₜ of the intermolecular bond in the structure-optimized surface adsorption structure or the absolute value of the adsorption energy E_{ads} of the molecule in the surface adsorption structure, in the order from the largest in the interaction index Iᵢₙₜ or the absolute value of the absolute value of the adsorption energy E_{ads} of the molecule.

By performing clustering to extract candidate molecule groups, the degree of selectivity of the molecular crystal for the molecule can be found from the degree of dispersion of the candidate molecule groups (see FIG. 9).

As the clustering method, a generally used method can be used, and as the clustering method, for example, the k-Means method, the k-Means++ method, or the Gaussian Mixture method is used.

Additionally, in the clustering step (step S211), the clustering unit 211 may visualize the extracted candidate molecule groups, perform nonlinear dimensionality reduction of the high-dimensional data to two dimensions or three dimensions, and then perform clustering. By calculating the distance between the molecular crystal and the molecule in the surface adsorption structure of each of the extracted candidate molecule groups, variables of the dimensions, of which the number corresponds to the number of extracted molecules (for example, 100 dimensions when 100 molecules are extracted) are obtained. The clustering unit 211 visualizes the extracted candidate molecule groups and performs nonlinear dimensionality reduction of the high-dimensional data into two dimensions or three dimensions, thereby facilitating appropriate clustering.

Examples of the method of visualizing the extracted candidate molecule groups include PCA and the like. In the clustering step (step S211), the clustering unit 211 can visualize the data of the extracted candidate molecule groups so that the scatter of points appears as large as possible with a smaller number of coordinate axes by using PCA.

As a method of performing the nonlinear dimensionality reduction of the high-dimensional data to two dimensions or three dimensions, for example, a t-SNE method of maintaining a relationship of an intermolecular distance between a molecular crystal and a molecule, a GTM method of maintaining a positional relationship between a molecular crystal and a molecule, or the like is used.

Next, the selection unit 212 selects a candidate molecule group from the multiple molecules (a selection step: step S212).

In the selection step (step S212), the selection unit 212 may select a suitable molecule group as the candidate molecule group according to the magnitude of the adsorptive property required for the molecule.

The method of selecting the molecule group is not particularly limited, and a general selection method may be used.

Next, the output unit 213 outputs the candidate molecule group by displaying or the like (an output step: step S213).

The screening method according to the first embodiment includes the first energy calculation step (step S203), the second energy calculation step (step S204), the surface adsorption structure search step (step S205), the surface adsorption structure optimization step (step S206), the third energy calculation step (step S207), the adsorption energy calculation step (step S208), the interaction index calculation step (step S209), the correlation diagram creation step (step S210), the clustering step (step S211), and the selection step (step S212). The screening method according to the first embodiment can select multiple molecules as the molecule group according to the adsorption strength of the molecule adsorbed to the molecular crystal in the selection step (step S212) by performing clustering based on the interaction index Iᵢₙₜ and the absolute value of the adsorption energy E_{ads} in the clustering step (step S211). With this, the screening method according to the first embodiment can extract a candidate molecule group having a suitable adsorption magnitude from among the multiple molecules. The adsorptive property between the molecular crystal and the molecule in the surface adsorption structure can be found from the interaction index Iᵢₙₜ and the absolute value of the adsorption energy E_{ads}. Therefore, the screening method according to the first embodiment can predict the adsorptive property of the molecule without directly obtaining the adsorption energy of the molecule adsorbed to the surface of the molecular crystal, and thus can extract a candidate molecule group having a suitable adsorption magnitude.

Here, in the screening method according to the first embodiment, the first acquisition step (step S201) may be performed after the second acquisition step (step S202) or may be performed simultaneously with the second acquisition step (step S202), and the order of the steps is not particularly limited.

Additionally, in the screening method according to the first embodiment, the first energy calculation step (step S203) may be performed after the second energy calculation step (step S204) or may be performed simultaneously with the second energy calculation step (step S204), and the order of the steps is not particularly limited.

### Second Embodiment

The screening method according to the second embodiment will be described. The screening method according to the second embodiment is a method using the screening device 20B having a configuration as illustrated in FIG. 10. In the screening method according to the second embodiment, in the screening device 20B having the configuration as illustrated in FIG. 10, the interaction index Iᵢₙₜ is predicted from the descriptor of the molecular structure of the prediction target molecule by the generated learned model M2, as the index representing the magnitude of the adsorptive property when the molecule is adsorbed to the molecular crystal, and a candidate molecule group is selected. The screening method according to the present embodiment is performed using the screening device 20B described above, and therefore, a part of the contents already described will be omitted.

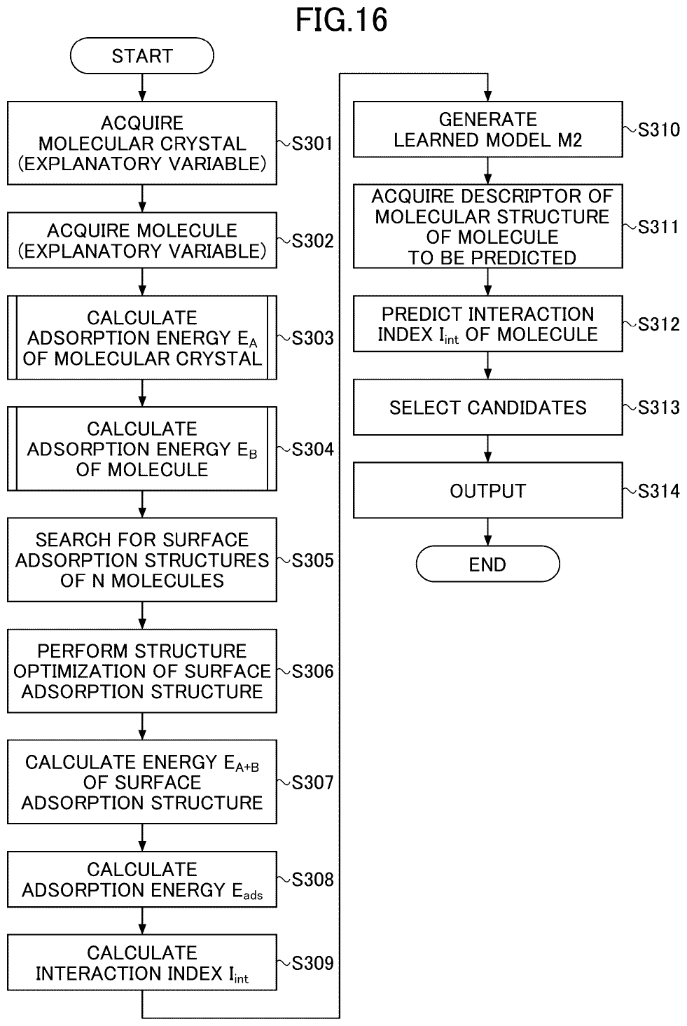

FIG. 16 is a flowchart illustrating the screening method according to the second embodiment. A first acquisition step (step S301), a second acquisition step (step S302), a first energy calculation step (step S303), a second energy calculation step (step S304), a surface adsorption structure search step (step S305), a surface-adsorption-structure structure optimization step (step S306), a third energy calculation step (step S307), the adsorption energy calculation step (step S308), and an interaction index calculation step (step S309) of the screening method according to the second embodiment illustrated in FIG. 16 are substantially the same as the first acquisition step (step S201) to the interaction index calculation step (step S209) of the screening method according to the first embodiment illustrated in FIG. 13, and thus the description thereof is omitted.

In the screening method according to the second embodiment, the learning model generation unit 221 generates the learned model M2 by using the descriptor of the molecular structure of the molecule (the explanatory variable) acquired in the second acquisition process (step S302) and the interaction index Iᵢₙₜ (the objective variable) calculated in the interaction index calculation step (step S309) (the learning model generation step: step S310).

In the learning model generation step (step S310), the learning model generation unit 221 generates the training dataset in which the descriptor of the molecular structure of the molecule (the explanatory variables) acquired in the second acquisition process (step S302) is associated with the interaction index Iᵢₙₜ (the objective variable) calculated in the interaction index calculation process (step S209), and performs learning by using the generated training dataset to generate the learned model M2.

As the learned model M2, the learned model M1 generated by the learning method according to the present embodiment described above can be used.

Next, the third acquisition unit 222 acquires the descriptor of the molecular structure of the prediction target molecule as the explanatory variable (a third acquisition step: step S311).

The information on the descriptor of the molecular structure of the prediction target molecule may be acquired from the third storage unit 303 of the storage unit 30. The third storage unit 303 stores the information on the descriptor of the molecular structure of the molecule, as with the second storage unit 112 described above. The information on the descriptor of the molecular structure of the molecule is substantially the same as the information of the descriptor of the molecular structure of the molecule acquired from the second storage unit 112 in the learning method described above, and thus the description thereof will be omitted.

Next, the prediction unit 223 predicts the interaction index Iᵢₙₜ by inputting the descriptor of the molecular structure of the prediction target molecule, acquired in the third acquisition process (step S311), by using the learned model M2 (a prediction step: step S312).

That is, in the prediction step (step S312), the prediction unit 223 inputs the descriptor of the molecular structure of the prediction target molecule, acquired in the third acquisition step (step S311), into the learned model M2, to output the interaction index Iᵢₙₜ of the prediction target, predicted by using the learned model M2, as the objective variable. As described above, the interaction index Iᵢₙₜ correlates with the adsorption energy of the molecule, and thus serves as the index representing the adsorption energy. Therefore, the prediction unit 223 can predict the adsorption energy of the prediction target molecule to the molecular crystal by predicting the interaction index Iᵢₙₜ corresponding to the descriptor of the molecular structure of the prediction target molecule from the learned model M2.

Next, the selection unit 212 selects a candidate molecule from among the multiple molecules (a selection step: step S313).

In the selection step (step S313), the selection unit 212 may select multiple suitable molecules as the candidate molecules in accordance with the magnitude of the adsorptive property required for the molecule.

The method of selecting the molecule is not particularly limited, and a general selection method may be used. As the selection method, for example, the molecule may be selected based on a condition that a predetermined number of molecules in the order from the largest in the interaction index Iᵢₙₜ or the absolute value of the adsorption energy E_{ads} are included, a condition that the interaction index Iᵢₙₜ or the absolute value of the adsorption energy E_{ads} is greater than or equal to a predetermined value, a condition that the interaction index Iᵢₙₜ or the absolute value of the adsorption energy E_{ads} is the highest, or the like.

Next, the output unit 213 outputs the selected molecule as the candidate molecule by displaying or the like (an output step: step S314).

The screening method according to the second embodiment includes the learning model generation step (step S310), the prediction step (step S312), and the selection step (step S313). In the prediction step (step S312), the interaction index Iᵢₙₜ of the prediction target molecule can be predicted as the index representing the magnitude of the adsorptive property from the descriptor of the molecular structure of the prediction target molecule by using the learned model M2. The screening method according to the second embodiment can predict the adsorptive property of the molecule without performing molecular simulation for obtaining an adsorption structure by predicting the interaction index Iᵢₙₜ of the prediction target. The screening method according to the second embodiment can select a candidate molecule from among multiple molecules based on the interaction index lint of the prediction target molecule in the selection step (step S313). The screening method according to the second embodiment can predict the adsorptive property of the molecule without directly obtaining the adsorption energy of the molecule adsorbed to the surfaces of the molecular crystal in the prediction step (step S312), and thus can select a candidate molecule from among multiple molecules.

Additionally, the screening method according to the second embodiment can predict the interaction index Iᵢₙₜ of the prediction target molecule by using the learned model M2 in the prediction process (step S312). The screening method according to the second embodiment can predict the magnitude of the adsorptive property of the molecule to the molecular crystal easily and in a shorter time by using the interaction index Iᵢₙₜ predicted using the learned model M2 in the prediction step (step S312), thereby shortening the time required to screen a molecule having a high adsorptive property to the molecular crystal and the like.

For example, the screening method according to the second embodiment can exhaustively screen, select, or design a new substance that has a strong hydrogen bond on the surface of a specific molecular crystal from a large number of compound groups. Therefore, the screening method according to the second embodiment can easily compare the adsorptive properties to the molecular crystal between different molecules, thereby performing molecular screening easily and in a shorter time.

Furthermore, the screening method according to the second embodiment can perform processing by using the machine learning potential 40 in at least one of the first energy calculation step (step S303), the second energy calculation step (step S304), the surface adsorption structure search step (step S305), or the surface-adsorption-structure structure optimization step (step S306). With this, the screening method according to the second embodiment can shorten the process time required to obtain an accurate adsorption structure between each molecule and the molecular crystal, for example, when it is desired to find a molecule exhibiting a strong adsorption state by hydrogen bonding from a large number of compound groups. Therefore, the screening method according to the second embodiment can shorten the screening time and can also cope with a wider range of molecule groups by enhancing versatility.

Here, in the screening method according to the second embodiment, the learning model generation step (step S310) may be performed after the third acquisition step (step S311) or may be performed simultaneously with the third acquisition step (step S311), and the order is not particularly limited.

In the above embodiments, the case where the adsorbent is the molecular crystal and the adsorbate is the molecule adsorbed to the molecular crystal has been described. However, the types of the adsorbent and the adsorbate are not limited thereto, and may be other substances. For example, the adsorbent may be a metal, a plastic, or the like, and the adsorbate may be the molecular crystal, the molecule, or the like.

As described above, the embodiments have been described, but the embodiments are presented as an example, and the present invention is not limited to the embodiments. The above-described embodiments can be implemented in various other forms, and various combinations, omissions, substitutions, changes, and the like can be made without departing from the spirit of the invention. These embodiments and modifications thereof are included in the scope and spirit of the invention, and are included in the invention described in the claims and the scope of equivalents thereof.

### EXAMPLES

In the following, the embodiments will be described more specifically with reference to the example and the comparative example, but the embodiments are not limited to these example and comparative example.

### Example 1

### (Preparation of Screening Device)

As the screening device, the screening device 20A having the configuration illustrated in FIG. 6 was prepared.

### (Preparation of Surface Adsorption Structure)

Three types of molecular crystals (acetanilide, ε-caprolactam, imidazole) and 2000 types of molecules to be adsorbed (molecules) were prepared using the screening device 20A having the configuration illustrated in FIG. 6. As the 2000 types of molecules, low molecules were randomly extracted from an existing database (QM9). Here, the random number seed was fixed to an arbitrary value to ensure consistency of the result. Three types of molecular crystals were adsorbed to each of the 2000 types of molecules, to prepare 6000 combinations of surface adsorption structures in total.

### (Calculation of Adsorption Energy E_{ads})

The energy E_{A} of the molecular crystal, the energy E_{B} of the molecule, and the energy E_{A+B} of the surface adsorption structure were calculated, and the adsorption energy E_{ads} of the molecule in the prepared surface adsorption structure was calculated based on the following equation (I).

$${E}_{ads} = {E}_{A + B} - ( {E}_{A} + {E}_{B} ) \qquad ( I )$$

### (Calculation of Interaction Index Iᵢₙₜ)

The interaction index Iᵢₙₜ in the case of focusing on the hydrogen bond of the prepared surface adsorption structure was calculated based on the following equation (1).

$$\begin{matrix}[ Eq . 2 ] & \\ {I}_{int} = \frac{A}{N} {\sum }_{i}^{N} exp ( - \frac{{R}_{i} - {R}_{0}}{{R}_{0}} ) & ( 1 )\end{matrix}$$

(where Iᵢₙₜ is the interaction index, A and R₀ are constants, N is the number of intermolecular bonds of interest at the interface with the adsorbent to which the adsorbate is surface-adsorbed, and Rᵢ is the distance of the intermolecular bonds of interest at the interface with the adsorbent to which the adsorbate is surface-adsorbed.)

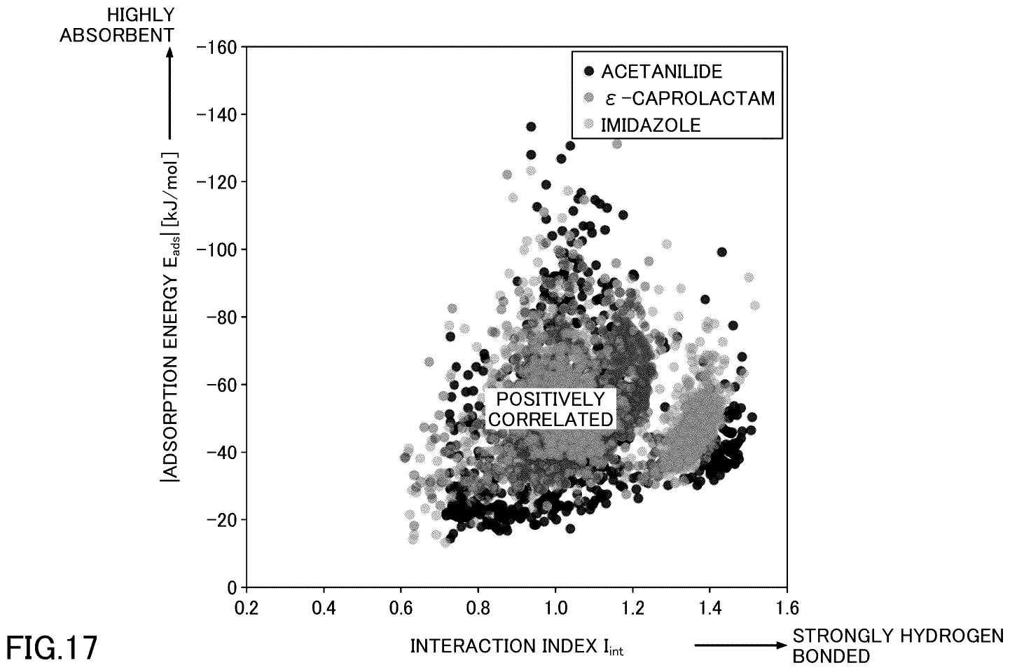

FIG. 17 is a correlation diagram in which the relationship between the interaction index Iᵢₙₜ and the absolute value of the adsorption energy E_{ads} of the 6000 surface adsorption structures are plotted. As illustrated in FIG. 17, it is confirmed that the absolute value of the adsorption energy E_{ads} tends to increase as the interaction index Iᵢₙₜ of the hydrogen bond of the molecule to the surface of the molecular crystal increases, and a positive correlation is indicated.

### (Clustering)

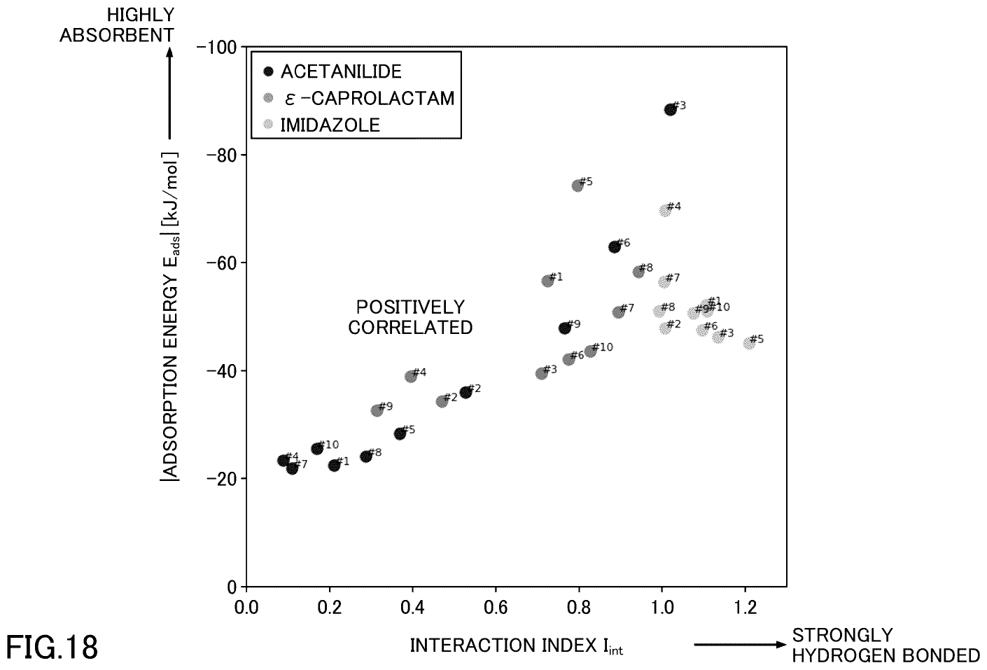

The distances between the molecular crystals and the molecules in the prepared surface adsorption structures were calculated, and the top 100 structures were extracted in the order from the shortest distance. This indicates that 100-dimensional variables are acquired by calculating the distances between the molecular crystals and the molecules in 100 surface adsorption structures. Next, the dimension was reduced from 100 dimensions to 2 dimensions by the t-SNE method, and clustering was performed by the k-means method, and molecules having similar properties were grouped by color. The number of clusters was 10. The clustering result is illustrated in FIG. 18.

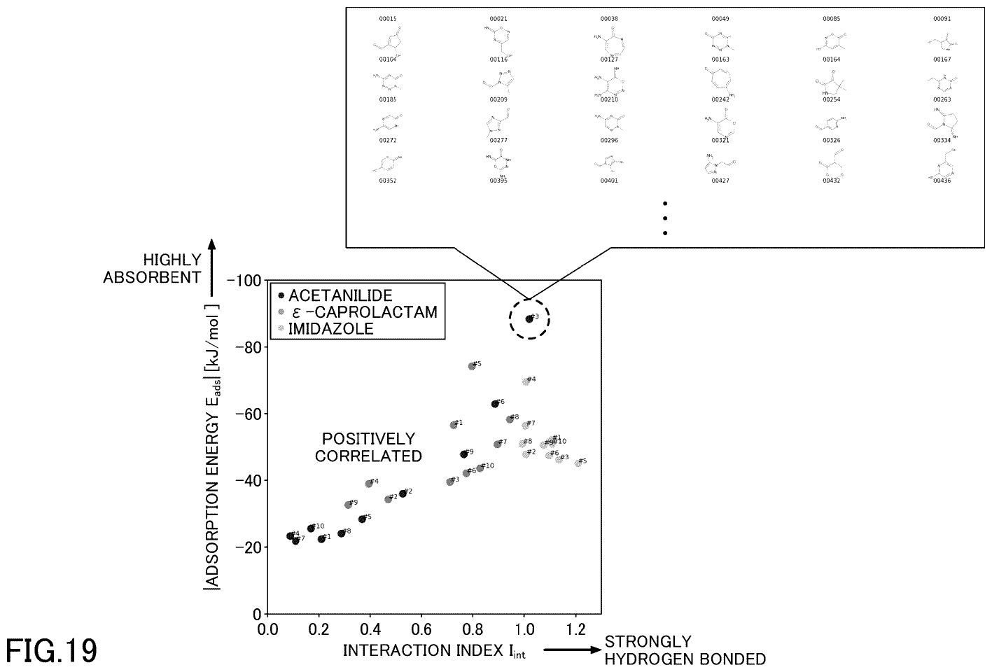

As illustrated in FIG. 18, it was confirmed that by clustering, a candidate molecule can be extracted as a group including multiple molecules, instead of extracting a specific single molecule. Therefore, it can be said that a molecule group having a strong hydrogen bond or a molecule group having a weak hydrogen bond can be extracted by clustering. For example, it was confirmed that when it is desired to select a molecule having a high hydrogen bond to acetanilide, the molecule group #3 can be selected. Additionally, as illustrated in FIG. 19, it was confirmed that multiple molecules belonging to the molecule group #3 can be extracted by selecting the molecule group #3.

Additionally, it was confirmed that, by clustering, the degree of selectivity for molecules can be grasped for each molecular crystal as illustrated in FIG. 18. For example, when the molecular crystal is imidazole, multiple molecule groups are concentrated in substantially one place, and thus it can be said that imidazole has low selectivity for molecules.

Therefore, the adsorptive property of each molecule can be confirmed for each molecular crystal, and thus it can be said that the molecule can be easily selected according to the type of the molecular crystal.

### Example 2

### (Preparation of Screening Device)

As the screening device, the screening device 20B having the configuration illustrated in FIG. 10 was prepared.

### (Preparation of Surface Adsorption Structure)

Using the screening device 20B having the configuration illustrated in FIG. 10, 2000 types of molecules were adsorbed on each of the three types of molecular crystals in the same manner as in Example 1, to prepare 6000 combinations of surface adsorption structures in total.

### (Generation of Learned Model)

From SMILES of the 2000 molecules, the descriptors thereof (the number of functional groups, the number of branches, the charge, and the like, 208 descriptors) were obtained using RDKit, and the correlation between 208 descriptors of the molecular structure of the molecule and the interaction index Iᵢₙₜ (the objective variable) was learned to generate the learned model. A gradient boosting regression tree was used as the learned model.

### (Calculation of Adsorption Energy E_{ads})

As in Example 1, the energy E_{A} of the molecular crystal, the energy E_{B} of the molecule, and the energy E_{A+B} of the surface adsorption structure were calculated, and the adsorption energy E_{ads} of the molecule in the prepared surface adsorption structure was calculated.

### (Prediction of Interaction Index Iᵢₙₜ)

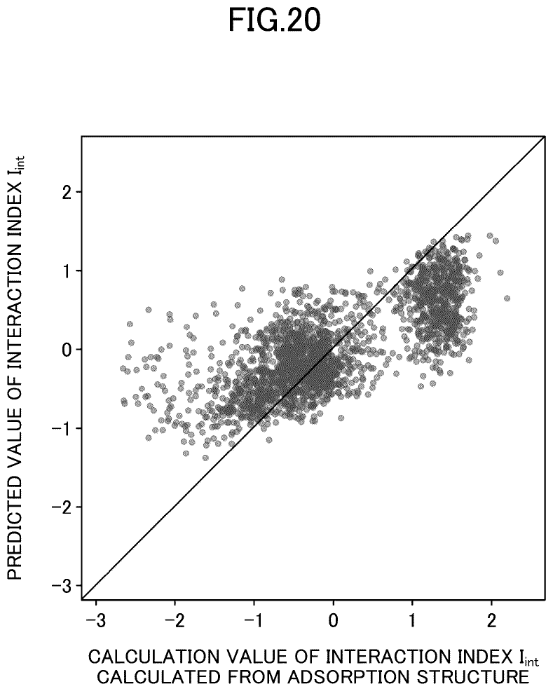

Using the generated learned model, the interaction index Iᵢₙₜ of the descriptors of the adsorption structure for each of the 2000 molecules is predicted. FIG. 20 indicates the correlation between the calculated value of the interaction index Iᵢₙₜ and the predicted value of the interaction index Iᵢₙₜ. As illustrated in FIG. 20, a positive correlation was observed between the calculated value of the interaction index Iᵢₙₜ and the predicted value of the interaction index Iᵢₙₜ.

Here, when the learned model was generated using the absolute value of the adsorption energy E_{ads} as the objective variable, instead of the interaction index Iᵢₙₜ, the coefficient of determination between the absolute value of the adsorption energy E_{ads} and the predicted absolute value of the adsorption energy E_{ads} was larger than that when the objective variable was the interaction index Iᵢₙₜ. Therefore, it was confirmed that the adsorption property by hydrogen bonding and the selectivity can be predicted more accurately by using the interaction index Iᵢₙₜ as the objective variable than by using the absolute value of the adsorption energy E_{ads}.

### (Prediction of Interaction Index Iᵢₙₜ Using Learned Model)

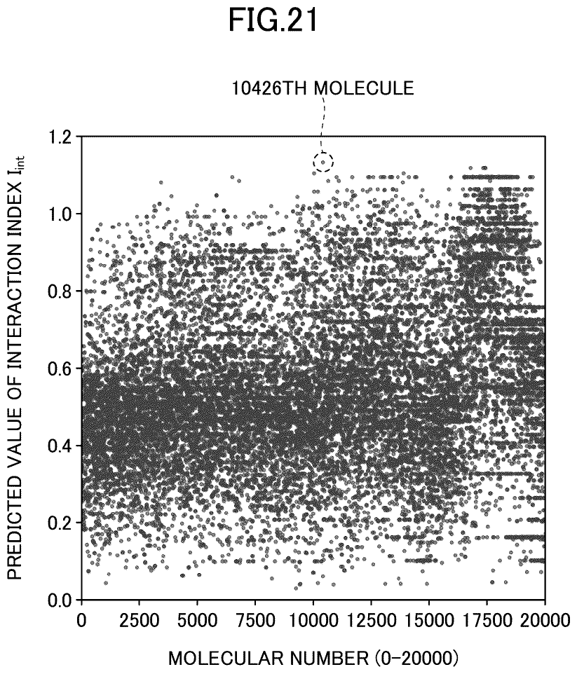

Using the generated learned model, the interaction index Iᵢₙₜ for the surface of the molecular crystal (acetanilide) was predicted, with respect to 20000 molecules that are not included in the training dataset (2000 molecules) used to generate the learned model. SMILES strings of the 20000 molecules were randomly extracted from the known database (QM9) such that the extracted SMILES strings do not overlap the training dataset, and the 208 descriptors of the adsorption structure of molecule were obtained by RDKit. The 208 descriptors of the adsorption structure of each molecule were input into the learned model, and the predicted values of the interaction index Iᵢₙₜ of the 20000 molecules were obtained. FIG. 21 indicates the relationship between a molecule number and the predicted value of the interaction index Iᵢₙₜ of the molecule.

As indicated in FIG. 21, the adsorptive property of each of the molecules to the surface of acetanilide was confirmed. Among them, the 10426th molecule has the greatest interaction index Iᵢₙₜ. Therefore, it is conceivable that the 10426th molecule can be selected as a molecule capable of increasing the adsorptive property to acetanilide because the hydrogen bond to the surface of acetanilide can be strengthened.

Therefore, the screening device of the present embodiment can predict the interaction index Iᵢₙₜ by inputting the descriptors of the adsorption structure of the molecule into the learned model, and thus can predict the adsorptive property of the adsorbate to the target adsorbent. The screening device of the present embodiment can screen a candidate adsorbate having an appropriate adsorptive property to the surface of the target adsorbent at high speed, and thus it is conceivable that the screening device of the present embodiment can be effectively used to screen an appropriate adsorbent substance according to the type of the adsorbent in the medical field, the industrial field, and the like.

Aspects of the embodiments of the present disclosure are as follows, for example.
<1> A learning device includes:
   a processor; and
   a memory storing program instructions that cause the processor to:
      generate a learned model by performing machine learning using a training dataset in which a descriptor of a molecular structure of an adsorbate to be adsorbed to an adsorbent is associated with an interaction index of one or more intermolecular bonds of interest between the adsorbate and the adsorbent.
<2> The learning device as described in <1>, wherein the interaction index is obtained by a following Equation (1):

$$\begin{matrix}[ Eq . 3 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$

where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.
<3> A learning model generated by using a training dataset in which a descriptor of a molecular structure of an adsorbate to be adsorbed to an adsorbent is associated with an interaction index of one or more intermolecular bonds of interest between the adsorbate and the adsorbent.
<4> The learning model as described in <3>, wherein the interaction index is obtained by a following Equation (1):

$$\begin{matrix}[ Eq . 4 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$

where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.
<5> A screening device includes:
   a processor; and
   a memory storing program instructions that cause the processor to:
      acquire a prediction target adsorbent and a descriptor of a molecular structure of a prediction target adsorbate to be adsorbed to the prediction target adsorbent; and
      predict an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.
<6> The screening device as described in <5>, wherein the interaction index is obtained by a following equation (1):

$$\begin{matrix}[ Eq . 5 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$

where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.
<7> The screening device as described in <5>, wherein the program instructions further cause the processor to:
   calculate energy E_{A} of the adsorbent;
   calculate energy E_{B} of the adsorbate; and
   calculate energy E_{A+B} of a surface adsorption structure in which the adsorbate is adsorbed to the adsorbent.
<8> The screening device as described in <7>, wherein the program instructions further cause the processor to calculate adsorption energy E_{ads} of the adsorbate from the energy E_{A} of the adsorbent, the energy E_{B} of the adsorbate, and the energy E_{A+B} of the surface adsorption structure.
<9> The screening device as described in <8>, wherein the program instructions further cause the processor to calculate the interaction index.
<10> The screening device as described in <9>, wherein the program instructions further cause the processor to generate the learned model by using the descriptor of the molecular structure of the adsorbate used to calculate the energy E_{B} and the calculated interaction index.
<11> The screening device as described in any one of <7> to <10>, wherein at least one of the calculating of the energy E_{A}, the calculating of the energy E_{B}, or the calculating of the energy E_{A+B} is performed using a machine learning potential.
<12> The screening device as described in any one of <5> to <11>, wherein either the adsorbent or the adsorbate is a molecular crystal.
<13> The screening device as described in any one of <5> to <12>, wherein the adsorbent is a molecular crystal and the adsorbate is a molecule.
<14> A non-transitory computer-readable recording medium having stored therein a screening program for causing a computer to:
   acquire a prediction target adsorbent and a descriptor of a molecular structure of a prediction target adsorbate to be adsorbed to the prediction target adsorbent; and
   predict an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.
<15> The screening program as described in <14>, wherein the interaction index is obtained by the following formula (1):

$$\begin{matrix}[ Eq . 6 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$

where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.
<16> A screening method comprising:
   acquiring a prediction target adsorbent and a descriptor of a molecular structure of a prediction target adsorbate to be adsorbed to the prediction target adsorbent; and
   predicting an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.
<17> The screening method as described in <16>, wherein the interaction index is obtained by the following equation (1):

$$\begin{matrix}[ Eq . 7 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$

where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.

What is claimed is:

1. A learning device comprising:
a processor; and
a memory storing program instructions that cause the processor to:
generate a learned model by performing machine learning using a training dataset in which a descriptor of a molecular structure of an adsorbate to be adsorbed to an adsorbent is associated with an interaction index of one or more intermolecular bonds of interest between the adsorbate and the adsorbent.

2. The learning device as claimed in claim 1, wherein the interaction index is obtained by a following Equation (1):
$$\begin{matrix}[ Eq . 1 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$
where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.

3. A learning method comprising:
generating a learning model by using a training dataset in which a descriptor of a molecular structure of an adsorbate to be adsorbed to an adsorbent is associated with an interaction index of one or more intermolecular bonds of interest between the adsorbate and the adsorbent.

4. The learning method as claimed in claim 3, wherein the interaction index is obtained by a following Equation (1):
$$\begin{matrix}[ Eq . 2 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$
where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.

5. A screening device comprising:
a processor; and
a memory storing program instructions that cause the processor to:
acquire a prediction target adsorbent and a descriptor of a molecular structure of a prediction target adsorbate to be adsorbed to the prediction target adsorbent; and
predict an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.

6. The screening device as claimed in claim 5, wherein the interaction index is obtained by a following equation (1):
$$\begin{matrix}[ Eq . 3 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$
where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.

7. The screening device as claimed in claim 5, wherein the program instructions further cause the processor to:
calculate energy E_{A} of the adsorbent;
calculate energy E_{B} of the adsorbate; and
calculate energy E_{A+B} of a surface adsorption structure in which the adsorbate is adsorbed to the adsorbent.

8. The screening device as claimed in claim 7, wherein the program instructions further cause the processor to calculate adsorption energy E_{ads} of the adsorbate from the energy E_{A} of the adsorbent, the energy E_{B} of the adsorbate, and the energy E_{A+B} Of the surface adsorption structure.

9. The screening device as claimed in claim 8, wherein the program instructions further cause the processor to calculate the interaction index.

10. The screening device as claimed in claim 9, wherein the program instructions further cause the processor to generate the learned model by using the descriptor of the molecular structure of the adsorbate used to calculate the energy E_{B} and the calculated interaction index.

11. The screening device as claimed in claim 7, wherein at least one of the calculating of the energy E_{A}, the calculating of the energy E_{B}, or the calculating of the energy E_{A+B} is performed using a machine learning potential.

12. The screening device as claimed in claim 5, wherein either the adsorbent or the adsorbate is a molecular crystal.

13. The screening device as claimed in claim 5, wherein the adsorbent is a molecular crystal and the adsorbate is a molecule.

14. A screening method comprising:
acquiring a prediction target adsorbent and a descriptor of a molecular structure of a prediction target adsorbate to be adsorbed to the prediction target adsorbent; and
predicting an interaction index of one or more intermolecular bonds of interest between the prediction target adsorbate and the prediction target adsorbent, using a learned model generated using a training dataset in which a descriptor of a molecular structure of an adsorbate to be adsorbed to an adsorbent is associated with an interaction index of one or more intermolecular bonds of interest between the adsorbate and the adsorbent.

15. The screening method as claimed in claim 14, wherein the interaction index is obtained by a following equation (1):
$$\begin{matrix}[ Eq . 3 ] & \\ {I}_{int} = \frac{A}{N} {\sum }_{i}^{N} exp ( - \frac{{R}_{i} - {R}_{0}}{{R}_{0}} ) , & ( 1 )\end{matrix}$$
where Iᵢₙₜ is the interaction index, A and R₀ are constants, N is a number of the one or more intermolecular bonds of the interest at an interface with the adsorbent to which the adsorbate is surface-adsorbed, and Rᵢ is a distance of the one or more intermolecular bonds of the interest at the interface with the adsorbent to which the adsorbate is surface-adsorbed.

16. The screening method as claimed in claim 14, further comprising:
calculating energy E_{A} of the adsorbent;
calculating energy E_{B} of the adsorbate; and
calculating energy E_{A+B} of a surface adsorption structure in which the adsorbate is adsorbed to the adsorbent.

17. The screening method as claimed in claim 16, further comprising calculating adsorption energy E_{ads} of the adsorbate from the energy E_{A} of the adsorbent, the energy E_{B} of the adsorbate, and the energy E_{A+B} of the surface adsorption structure.

18. The screening method as claimed in claim 17, further comprising calculating the interaction index.

19. The screening method as claimed in claim 18, further comprising generating the learned model by using the descriptor of the molecular structure of the adsorbate used to calculate the energy E_{B} and the calculated interaction index.

20. The screening method as claimed in claim 16, wherein at least one of the calculating of the energy E_{A}, the calculating of the energy E_{B}, or the calculating of the energy E_{A+B} is performed using a machine learning potential.
